(19) United States
(12) Patent Application Publication
(10) Pub. No.: US 2024/0335594 A1
(43) Pub. Date: Oct. 10, 2024

(54) METHOD FOR CONSOLIDATING AN ALGINATE/GELATIN HYDROGEL

(71) Applicants: UNIVERSITE CLAUDE BERNARD LYON 1, Villeurbanne (FR); CENTRE NATIONAL DE LA RECHERCHE SCIENTIFIQUE (CNRS), Paris (FR); ECOLE SUP CHIMIE PHYS ELECTRONIQ LYON, Villeurbanne (FR); LAB SKIN CREATIONS, Lyon (FR)

(72) Inventors: Christophe MARQUETTE, Villeurbanne (FR); Emma PETIOT, Lyon (FR); Laura CHASTAGNIER, Villeurbanne (FR); Audrey CHERBLANC, Sarcey (FR); Morgan DOS SANTOS, Saint Verand (FR); Amélie THEPOT, Lyon (FR); Baptiste GODET, Perros Guirec (FR)

(73) Assignees: UNIVERSITE CLAUDE BERNARD LYON 1, Villeurbanne (FR); CENTRE NATIONAL DE LA RECHERCHE SCIENTIFIQUE (CNRS), Paris (FR); ECOLE SUP CHIMIE PHYS ELECTRONIQ LYON, Villeurbanne (FR); LAB SKIN CREATIONS, Lyon (FR)

(21) Appl. No.: 18/573,879

(22) PCT Filed: Jun. 24, 2022
(86) PCT No.: PCT/FR2022/051264

§ 371 Date: Dec. 22, 2023

(30) Foreign Application Priority Data
Jun. 25, 2021 (FR) 2106826

(51) Int. Cl. A61L 27/52; A61L 27/26; A61L 27/38

(52) U.S. Cl. CPC A61L 27/52; A61L 27/26; A61L 27/3834

(57) ABSTRACT

A process for consolidating a hydrogel including alginate and gelatin, which includes a step of consolidation by crosslinking of the hydrogel using a suitable solution, in order to give it particularly advantageous mechanical properties. Also, the use of this crosslinking solution for consolidating the structure of a hydrogel including alginate and gelatin, and to a hydrogel obtained by this process. Additionally, a process for consolidating the structure of a hydrogel including alginate and gelatin, this process including preparing the hydrogel, shaping it, then bringing it into contact with suitable consolidation agents. Further, the hydrogel obtained by this process. In all these aspects, the hydrogel can further include fibrinogen and/or living cells.

### FIELD OF INVENTION

The present invention relates to the field of the manufacture of materials in general, and in particular to bio-materials or bio-compatible materials.

The present invention relates to a method for consolidating a hydrogel comprising alginate and gelatin, which includes a step of consolidation by crosslinking the hydrogel using a suitable solution, in order to give it particularly advantageous mechanical properties. The use of this crosslinking solution to consolidate the structure of a hydrogel comprising alginate and gelatin also constitutes another aspect of the present invention. The hydrogel obtained by this method is also another aspect of the present invention.

The present invention also relates to a method for consolidating a hydrogel comprising alginate and gelatin, said method comprising the preparation of the hydrogel, its shaping, then contacting it with at least one divalent cation, preferably calcium, and transglutaminase. The hydrogel obtained by this method is also another aspect of the present invention.

In all the aspects of this invention, the hydrogel may further comprise fibrinogen and/or living cells.

### BACKGROUND OF INVENTION

Hydrogel-based structures comprising alginate and gelatin are known from the prior art, but they lack satisfactory mechanical strength, because these constituents most often have limited elasticity (low Young's modulus, in particular), which makes the resulting structures difficult to handle.

The international application published under number WO2017115056 describes the manufacture of body substitutes based on hydrogel comprising alginate, gelatin and fibrinogen, which are then treated with a crosslinking solution containing calcium and thrombin.

The mechanical properties of the structures obtained in the prior art nevertheless remain insufficient to make them suitable for being manipulated. In addition, in the case of structures intended to be implanted in the human or animal body, and if necessary sutured, the methods of the prior art do not make it possible to obtain structures having adequate mechanical properties, and whose degradation in contact with living cells or tissues is not too fast.

Moreover, when the structures are likely to be cellularized during their manufacture, there is a need to be able to maintain the survival of the cells. Thus, the method for preparing such structures must be compatible with the presence of living cells and their life support.

### SUMMARY

One of the aims of the invention is to overcome these drawbacks of the prior art, and to enable in particular to produce hydrogels, cellularized or not, which have particularly advantageous and innovative characteristics, in particular in terms of (i) mechanical strength of the constituents, (ii) stability over time, (iii) flexibility, (iv) tear and impact resistance, and (v) colonization by the cells.

For this purpose, it is proposed to provide, according to a first embodiment of the present invention, a method for consolidating a hydrogel comprising alginate and gelatin, said method comprising the preparation of the hydrogel, then contacting it with a consolidation solution comprising at least one divalent cation, preferentially calcium, and transglutaminase, and optionally one or more other divalent cations.

Optionally, the consolidation solution may also contain thrombin if, in addition to alginate and gelatin, the hydrogel contains fibrinogen. Moreover, the consolidation step is compatible with the fact that the hydrogel can contain living cells.

According to another aspect of the present invention, it is described the use of a solution as described above to consolidate the structure of a hydrogel comprising alginate and gelatin, which has a particular advantage when preparing a hydrogel intended to be implanted in the human or animal body, i.e. a body implant.

This use may also be used to consolidate a hydrogel further comprising fibrinogen. Moreover, the consolidation can also be carried out on a hydrogel further comprising living cells.

In summary, the present invention relates to a method for consolidating a hydrogel comprising alginate and gelatin, said method comprising the preparation of the hydrogel, contacting it with a consolidation solution comprising at least one divalent cation, preferentially calcium, and transglutaminase, and optionally one or more other divalent cations.

Preferably, the contact of the hydrogel with the consolidation solution is carried out by immersion of the hydrogel, preferably total immersion of the hydrogel, in the consolidation solution, or by spraying the hydrogel with the consolidation solution.

Preferably, the divalent cation(s) is/are chosen from the group comprising calcium, strontium and barium.

Preferably, the hydrogel comprises from 0.5 to 3% of alginate and from 1 to 17.5% of gelatin.

Preferably, the hydrogel further comprises fibrinogen and in that the consolidation solution further comprises thrombin.

Preferably, the hydrogel comprises up to 2% of fibrinogen.

Preferably, the consolidation solution comprises:
from 0.003 to 30% of divalent cation,
from 0.004 to 16% of transglutaminase, and optionally
from 2 to 25 U/ml of thrombin.

Preferably, the hydrogel further comprises living cells integrated during hydrogel preparation prior to its consolidation.

Preferably, the contact with the consolidation solution is carried out:
at a temperature ranging from 15 to 40° C. and/or
for a period ranging from 30 minutes to 6 h.

Preferably, the hydrogel is shaped prior to its consolidation, preferentially said shaping is done by extrusion of material, even more preferentially said shaping is done by molding or additive manufacturing technique.

According to another aspect, the present invention also relates to the use of a solution comprising a divalent cation, preferably calcium, and transglutaminase, and optionally one or more other divalent cations, for consolidating a hydrogel comprising alginate and gelatin.

According to another aspect, the present invention also relates to a hydrogel comprising consolidated alginate and gelatin, intended to be implanted in a human subject, the hydrogel being obtainable by a consolidation method as described previously.

It is also proposed to provide, according to a second embodiment of the present invention, a method for consolidating a hydrogel comprising alginate and gelatin, said method comprising, in order, the following steps:
preparation of the hydrogel,
shaping the prepared hydrogel,
contacting the hydrogel with at least one divalent cation, preferably calcium, and transglutaminase, and optionally one or more other divalent cations.

Preferably, said hydrogel preparation does not require the shaping of one or more components prior to the preparation of the hydrogel.

Preferably, said hydrogel preparation does not require the addition of fibers.

Preferably, the divalent cation(s) is/are chosen from the group comprising calcium, strontium and barium.

According to another aspect, the present invention relates to a hydrogel comprising consolidated alginate and gelatin, intended to be implanted in a human subject, the hydrogel being obtainable by a consolidation method as described previously.

### Definitions

In the present invention, the terms below are defined as follows:
“Crosslinking agent”, in the context of the present invention, refers to an agent capable of crosslinking the components of the hydrogel, in particular alginate, gelatin and fibrinogen.
“Concomitant”: in the context of the present invention, refers to events taking place at the same time. Thus, the concomitant contact of the hydrogel with calcium and transglutaminase means that the hydrogel comprising alginate and gelatin is brought into contact, at the same time, with calcium and transglutaminase, thus allowing concomitant crosslinking of alginate and gelatin.
“Fibers”, in the context of the present invention, refers to any element of filamentous appearance, generally in the form of bundles. Examples of fibers are given below.
“Shape”, in the context of the present invention, consists in giving the shaped element, a particular shape, structure and/or architecture. When it is the shaping of a hydrogel, this consists, for example, in giving it a particular shape, structure and/or architecture, particularly suited to the destination of the hydrogel once consolidated. When it is the shaping of one or more components of the hydrogel during its preparation, this consists, for example, in giving them a particular structure, such as a structure in the form of fibers, during hydrogel preparation.
“Crosslinking” or “Consolidate”, in the context of the present invention, are equivalent terms and designate the fact of consolidating the hydrogel by crosslinking alginate, gelatin and/or fibrinogen.
“Sequentially”, in the context of the present invention, refers to events that do not take place at the same time. Thus, the sequential contact of the hydrogel with calcium and transglutaminase means that the hydrogel is brought into contact with calcium, then transglutaminase or with trasnglutaminase and then calcium.
“Consolidation solution” or “crosslinking solution”, in the context of the present invention, are equivalent terms and refer to the solution used in the method according to the invention for consolidating the hydrogel by crosslinking.

### DETAILED DESCRIPTION

The present invention provides a method for preparing hydrogels comprising alginate and gelatin, cellularized or not. By crosslinking the hydrogel constituents using at least one divalent cation, preferably calcium, and transglutaminase, the structure of the hydrogel will be consolidated, giving it particularly advantageous mechanical characteristics, in particular in terms of mechanical strength of the constituents, stability over time and remarkable tearing and impacts resistance.

According to a first embodiment, the present invention relates to a method for consolidating a hydrogel comprising alginate and gelatin, said method comprising the preparation of the hydrogel, then contacting it with a consolidation solution comprising at least one divalent cation, preferentially calcium, and transglutaminase, and optionally one or more other divalent cations.

In the context of the present invention, said consolidation solution can be obtained by alternative but nevertheless equivalent methods. The consolidation solution can be obtained by adding the various elements, i.e. at least one divalent cation, preferably calcium, and transglutaminase, and optionally one or more other divalent cations, in a same solution, or by mixing at least two solutions: a solution comprising at least one divalent cation, preferably calcium, and optionally one or more other divalent cations, and a solution comprising at least transglutaminase.

Alginate is a linear polysaccharide extracted from marine algae, mainly from the species of brown alga Phaeophyceae. This biocompatible polymer is composed of homopolymeric blocks of acid 1,4β-D mannuronic acid (M) and its epimeric acid C-5α-L guluronic acid (G). This biopolymer consists of sequences of M-blocks, G-blocks, intercalated with sequences of MG-blocks. Only the G units appear to be involved in intermolecular crosslinking during polymerization. Sodium alginate is widely used as a hydrogel.

Gelatin is a macromolecule derived from collagen which contains bioactive sequences such as the RGD (arginine-glycine-aspartic acid) motif for cell adhesion. It is obtained by denaturing the native triple helix structure of collagen via an acid treatment (type A gelatin), or alkaline (type B gelatin). The amino acid composition of gelatin is similar but different from that of collagen following denaturation (deamination of glutamine to glutamic acid in the type B gelatin manufacturing process). The structure of gelatin changes during gelation.

Preparation of hydrogels is well known in the art (E M Ahmed; Journal of Advanced Research, 2015, 6, 105-121), as well as polymerization and crosslinking of alginate and gelatin (Chen Q, Tian X, Fan J, Tong H, Ao Q, Wang X An Alginate/Gelatin Network for Three-Dimensional (3D) Cell Cultures and Organ Bioprinting. Molecules. 2020; 25 (3): 756)).

Regarding alginate, and based on what is mentioned above, an alginate enriched in M unit will be more flexible because the chain will have a more linear configuration, while a gel containing more G units will be more rigid because more polymerized. In the context of the present invention, the alginate used has, for example, a M/G ratio comprised between 1 and 2, in particular between 1 and 1.9 or between 1 and 1.5. In the context of the present invention, the alginate used has, for example, a M/G ratio of 1.9.

Preferably, the gelatin contained in the hydrogel is type A.

In the context of the present invention, the hydrogel is preferably prepared from an alginate solution to which a gelatin solution is added, or vice versa, from a gelatin solution to which an alginate solution is added, so as to obtain a hydrogel comprising from 0.5 to 3% of alginate and from 1 to 17.5% of gelatin, and even more preferably from 1 to 2.5% alginate and 2 to 10% of gelatin. Advantageously, the hydrogel comprises 2% of alginate and 5% of gelatin.

Unless otherwise indicated, the percentages mentioned in the present description are expressed by mass/volume of the total composition.

Preferably, when the hydrogel to be consolidated contains alginate and gelatin, these constituents are present in a mass ratio ranging from 1:0.3 to 1:35, and most particularly in a mass ratio of 1:2.5, respectively.

The consolidation solution preferably comprises:
from 0.003 to 30% of divalent cation(s),
from 0.004 to 16% of transglutaminase.

More preferably, the consolidation solution comprises from 1 to 6% of divalent cation(s), and advantageously 3% of divalent cation(s).

More preferably, the consolidation solution comprises from 1 to 10% of transglutaminase, and advantageously 4% of transglutaminase.

The transglutaminase enzyme (TAG) is an extracellular aminoacyltransferase. It is a monomeric protein which comprises a single cysteine catalytic residue (active site).

In the context of the invention, the consolidation solution preferably contains type 2 transglutaminase. This TAG is produced commercially as a recombinant microbial protein by fermentation of the microorganism Streptoverticillium moboarense. The consolidation solution used in the context of the invention may also contain several TAGs.

Any other divalent cation, preferably non-toxic divalent cation, can be used in the context of the method according to the invention.

For example, the divalent cation(s) is/are chosen from the group comprising or consisting of calcium, strontium, barium, zinc, copper, iron and nickel. Preferably, the divalent cation(s) is/are chosen from the group comprising or consisting of calcium, strontium and barium. Preferably, the divalent cation is calcium, but it may also be strontium or barium.

It may also be envisaged to use several divalent cations in a mixture. Moreover, the divalent cation(s) are present as salts in the consolidation solution. Any salt can be used, and preferably anhydrous salts.

Preferably, the consolidation solution comprises only a divalent cation, preferably calcium, and transglutaminase. Advantageously, if one or more divalent cation(s) other than calcium was/were present, it would preferably be a single other divalent cation, and in particular barium.

Preferably, the consolidation solution contains calcium chloride as the sole divalent cation.

According to the present invention, the simultaneous crosslinking of the hydrogel components, i.e. the transformation into a three-dimensional polymer from a linear polymer, is carried out by contacting it with a consolidation solution in which (i) the divalent cation, preferably calcium, or the other divalent cation(s) allow(s) the crosslinking of alginate and (ii) transglutaminase induces the enzymatic crosslinking of gelatin.

Preferably, during the consolidation process according to the invention, the contact of the hydrogel with the consolidation solution is carried out by immersion, during which the hydrogel is immersed in its entirety in the consolidation solution. It is also referred here to a consolidation bath. The contact of the hydrogel with the consolidation solution can also be carried out by imbibition, by spraying, using a drip, trickling, or similar system. The contact preferably refers to the entire hydrogel. Preferably, the hydrogel is entirely immersed in the consolidation solution.

The hydrogel to be consolidated according to the present invention may further comprise fibrinogen, in addition to alginate and gelatin.

The fibrinogen monomer is composed of two repetitions of three α, β and γ chains connected by a central E domain and two fibrinopeptides A and B (FpA, FpB) connecting the α chains to the E domain. It has a large number of cell adhesion motifs and thus allows an increased development of cells within the hydrogel.

Preferably, the hydrogel prepared to be consolidated according to the method of the invention is composed of alginate and gelatin, or alternatively of alginate, gelatin and fibrinogen, without any other constituent capable of forming a gel.

Preferably, when the hydrogel comprises fibrinogen, in addition to alginate and gelatin, it is prepared so as to contain up to 6% of fibrinogen, and in particular from 0.0001% to 6% of fibrinogen, and in particular 2% of fibrinogen.

Also preferably, when the hydrogel contains alginate, gelatin and fibrinogen, these constituents are present in a mass ratio ranging from 1:0.3:0.00003 to 1:35:12, and most particularly in a mass ratio of 1:1:2.5, respectively.

When the hydrogel contains fibrinogen in addition to alginate and gelatin, the consolidation solution used in the method according to the invention further comprises thrombin, i.e. thrombin in addition to calcium and TAG, and optionally one or more other divalent cations.

In the context of the present invention, said consolidation solution further comprising thrombin can be obtained by alternative but nevertheless equivalent methods. The consolidation solution can be obtained by adding the different elements, i.e. at least one divalent cation, preferentially calcium, transglutaminase, thrombin, and optionally one or more other divalent cations, in the same solution, or by mixing at least three solutions: a solution comprising at least one divalent cation, preferably calcium, and optionally one or more other divalent cations, a solution comprising at least transglutaminase, and a solution comprising thrombin. It is also possible for the thrombin to be integrated into the solution comprising at least one divalent cation or in the solution comprising transglutaminase. In this case, said consolidation solution can be obtained by mixing at least two solutions: i) a solution comprising at least one divalent cation, preferably calcium, and optionally one or more other divalent cations, and thrombin, and a solution comprising transglutaminase, or ii) a solution comprising at least one divalent cation, preferably calcium, and optionally one or more other divalent cations, and a solution comprising transglutaminase and thrombin.

In addition to what is mentioned above on the crosslinking of alginate and gelatin, thrombin, in this case, crosslinks the fibrinogen to fibrin. Fibrin is a natural biological polymer resulting from a polymerization mimicking the last step of the coagulation cascade, when thrombin acts on fibrinogen. Thrombin will first cleave the fibrinopeptide A, resulting in the formation of a protofibril. Cleavage of the fibrinopeptide B leads to the release of α chains and subsequent lateral polymerization of fibrinogen to form fibrin.

When the hydrogel to be consolidated by the method of the invention comprises fibrinogen, the consolidation solution used comprises thrombin, in addition to calcium and TAG, and optionally one or more other divalent cations. In this case and preferably, the consolidation solution comprises:
from 2 to 25 U/ml of thrombin,
from 0.003 to 30% of divalent cation(s), and
from 0.004 to 16% of transglutaminase.

As above, more preferably, the consolidation solution comprises from 1 to 6% of divalent cation(s), and advantageously 3% of divalent cation(s).

More preferably, the consolidation solution comprises from 1% to 10% % of transglutaminase, and advantageously 4% of transglutaminase.

Even more preferably, when the consolidation solution further comprises thrombin, the latter is preferably present from 2 to 10 U/ml.

Even more preferably, the consolidation solution comprises 4 U/ml of thrombin, 3% of calcium, and 4% of transglutaminase.

In the context of the invention, alginate, gelatin and, where appropriate, fibrinogen, which are used are chosen from those which have the most similar characteristics to the following:
alginate: viscosity of 130-300 mPa·s for a 2% solution;
gelatin: type A, porcine, degree of bloom 280 (force or resistance to depression);
fibrinogen: human, coagulable proteins level ≥91 mg/mL;
thrombin: human, activity ≥500 U/mL.

The hydrogels advantageously contain alginate, gelatin and optionally fibrinogen as natural components of the hydrogel.

However, other natural components may or may not be present in the hydrogels, such as: chitin, chitosan, cellulose, agarose, chondroitin sulfate, hyaluronic acid, glycogen, starch, pullulan, carrageenan, heparin, collagen, albumin, fibrin, fibroin, dextran, xanthan, gellan, any component extracted from extracellular matrix such as collagens, laminin, Matrigel-type proteoglycans, GelMa-type methacrylate gelatin.

In addition to the natural components, and in particular those listed above, the hydrogels of the present invention may also or may not contain synthetic components, such as polyolefins (PE, PP, PTFE, PVC), silicone (PDMS), polyacrylates (PMMA, pHEMA), polyester (PET, dacron, PGA, PLLA, PLA, PDLA, PDO, PCL), polyethers (PEEK, PES), polyamides, polyurethanes, PEG, pluronic F127.

Textile fibers of natural or synthetic origin may or may not be present in the hydrogels composition.

Examples of fibers of natural origin include, but are not limited to, cellulose fibers.

Examples of synthetic fibers include, but are not limited to, polyester fibers, nylon fibers, polyethylene fibers, polypropylene fibers, and acrylic fibers.

The hydrogel may further comprise living cells.

In the context of the present invention, said hydrogel may comprise alginate, gelatin, optionally fibrinogen, and optionally living cells. In the context of the present invention, said hydrogel may consist of alginate, gelatin, optionally fibrinogen, and optionally living cells.

These living cells may be of any type, except human embryonic stem cells obtained by destruction of an embryo, and preferably several types of living cells may coexist. The living cells are preferably chosen from cells of epithelial tissue, connective tissue, adipose tissue, endothelial tissue, and in particular from fibroblasts, keratinocytes, stem cells of adipose tissue, adipocytes, melanocytes, endothelial cells, macrophages, leukocytes, etc. In this implementation of the method of the invention, the cells are therefore manipulated under conditions that the person skilled in the art is able to determine, to maintain their viability and their proliferation, and ideally their differentiation.

These cells can be integrated during the preparation of the hydrogel when alginate, gelatin, and optionally fibrinogen solutions, are mixed to prepare the hydrogel, before its consolidation. It is then referred to a cellularized hydrogel. They may also be added to the consolidation solution, or following the consolidation of the hydrogel. Preferably, the hydrogel to be consolidated according to the invention comprises living cells which are integrated during the preparation of the hydrogel before its consolidation. For example, the living cells can be suspended in the fibrinogen solution, to which alginate and gelatin are added, in one or more steps, so as to obtain a hydrogel comprising the amounts of alginate/gelatin/fibrinogen mentioned above.

To illustrate different implementations of the present invention, the following sequences are preferably used to prepare the hydrogel to be consolidated according to the method of the invention:
(i) alginate is added to the suspension of living cells, then gelatin is added;
(ii) alginate is added to fibrinogen, then gelatin is added;
(iii) the living cells are suspended in fibrinogen, then alginate is added, and then gelatin.

Advantageously, the sequence (iii) is used to prepare a cellularized hydrogel according to the invention.

It is also possible that the hydrogel does not comprise living cells, it is therefore in this case acellular.

In these particular sequences, the ranges preferences for each of the constituents mentioned above also apply. It is also possible to prepare the hydrogel to be consolidated in a single step, i.e. by mixing all the constituents at the same time, whether this is alginate and gelatin, and optionally fibrinogen and/or living cells, where appropriate. Preferably, the consolidation step which consists in contacting the hydrogel with the consolidation solution is carried out at a temperature ranging from 15 to 40° C., and preferably from 20 to 40° C. and even more preferably from 21 to 37° C.

Preferably, this consolidation step is carried out for a period ranging from 10 minutes to 6 h, preferentially ranging from 30 minutes to 6 h, and ideally for 1 h to 3 h.

Thus, preferably, the consolidation step is carried out at 37° C. for 1 h 30.

Preferably, the hydrogel consists of alginate and gelatin, or of alginate, gelatin and fibrinogen, and the consolidation step is carried out at 37° C. for 1 h 30.

Preferably, the hydrogel consists of alginate and gelatin, or of alginate, gelatin and fibrinogen, and the consolidation step is carried out by total immersion of the hydrogel in the consolidation solution at 37° C. for 1 h 30.

In the context of the consolidation method of the invention, the latter may comprise a step of shaping the hydrogel once prepared, and prior to its consolidation.

The hydrogels as described in detail above, once prepared, can therefore be shaped, before consolidation, by various methods well known to those skilled in the art allowing a volume structuring (3D in particular), and in particular by adding or agglomerating material by stacking layers or successive deposits. Thus, the hydrogels as described above can be obtained by an additive manufacturing method.

Among these methods, we may mention in particular methods by injection, by extrusion, and in particular molding, or additive manufacturing, in particular 3D printing. Thus, the hydrogels as described above may be obtained by extrusion of material, preferably by a molding technique or by additive manufacturing, in particular 3D printing.

In particular, the person skilled in the art will take care to choose a method which allows the shaping of high-viscosity materials, since a hydrogel constituted solely of alginate and gelatin may have a viscosity ranging from 50 to 6000 Pa·s, for a measurement at a temperature of 5 to 45° C.

In the context of the invention, the hydrogel which is consolidated according to the consolidation method is preferably shaped before consolidation, preferably by a 3D printing technique.

As indicated previously, the implementation of the method according to the invention enables to give the hydrogel, once consolidated, advantageous mechanical properties, which are particularly suitable for the provision of a hydrogel intended to be implanted in the human or animal body, i.e. a body implant.

To do this, shaping the hydrogel, prior to its consolidation, using 3D printing offers the advantage of being able to prepare a custom structured hydrogel, whose dimensions and/or filling/porosity are defined with regard to the needs of the body intended to receive the body implant and the role/function that it should play in this recipient organism. Indeed, porosity of the implant is a key parameter to be adjusted according to the tissues or organs to be replaced and/or augmented. Porosity reflects the empty space present in the implant which can be adapted to provide more or less material and thus conferring a certain mechanical strength as close as possible to that of the native tissue of the implantation zone. In particular, it can be expressed in two different but correlated and therefore equivalent or alternative ways: size of the pores expressed in micrometers and/or hydrogel filling rate expressed as percentage (volume of hydrogel/total volume of the implant).

To consolidate a hydrogel containing living cells having such a destination (cellularized hydrogel or cellularization subsequent to the manufacture of the hydrogel), it is moreover particularly advantageous in terms of safety that the living cells are autologous cells, i.e. cells originating from the recipient organism.

Thus, in an exemplary embodiment, the hydrogel described previously enables to obtain a three-dimensional body implant which comprises one or more zones each having an overall porosity comprised between 100 μm and 10000 μm, while having a mechanical strength of 1 kPa to 1000 kPa. The overall porosity of a porous zone corresponds to an average of pore sizes measured in the porous zone.

The pores of the porous zone may have homogeneous pore sizes, i.e. differing from each other by no more than 15%.

The pores of the porous zone may be homogeneously, i.e. evenly distributed.

The pores of the porous zone may extend along central axes having respectively homogeneous orientations, i.e. differing from each other by not more than 20°. The central axes of the pores of the porous zone may be arranged with homogeneous spacings, i.e. not differing from one another by more than 15%.

The pores of the porous zone may each have homogeneous geometries, i.e., the contours of which may be superimposed with more than 50% of the portions overlapping or being parallel.

The pores of the porous zone may be separated from each other by material strand having respectively homogeneous thicknesses, i.e. not differing from each other by more than 15%.

In particular, the implant may comprise at least two porous zones in which the pores have different pore sizes and/or shapes.

The porous zones may be arranged to form a gradient of pore sizes distributed across the implant, with the porous zones following one another along a gradient direction in an order selected from an ascending order and a descending order of pore sizes.

In particular, the implant may comprise:
a first porous zone forming a base representing 5% to 40%, preferably 20% to 40%, of a total volume of the implant, and having a pore size comprised between 500 micrometers and 5000 micrometers, in particular 250 micrometers to 800 micrometers,
a second porous zone forming a core representing 20% to 70%, preferably 30% to 50%, of the total volume of the implant and having a pore size comprised between 500 micrometers and 2500 micrometers, in particular 100 micrometers to 250 micrometers,
a third porous zone forming a shell representing 5% to 40%, preferably 10% to 40%, of the total volume of the implant, and having a pore size comprised between 1000 micrometers and 10000 micrometers, in particular 1000 micrometers to 2500 micrometers.

The implant may also comprise at least one non-porous zone, the non-porous zone having a filling rate greater than 99%.

The at least one non-porous zone may comprise a perimeter surrounding the porous zone.

The porous zone(s) may cover an essential part of the implant, i.e. at least 50%, preferably at least 75%, in particular at least 90%, for example at least 95%.

The implant may consist of a plurality of layers each having a mesh consisting of a plurality of meshes, the layers being stacked on top of each other in such a way that the meshes form the pores.

The meshes of each layer may have homogeneous mesh sizes, i.e., differing by no more than 15% from one another.

The meshes in each layer may be homogeneously, i.e., evenly distributed.

The meshes of each layer may extend around central mesh axes having respectively homogeneous orientations, i.e. not differing from each other by more than 20°.

The central mesh axes of the meshes of each layer may be arranged with homogeneous spacings, i.e. not differing by more than 15% relative to one another.

The meshes of each layer may have homogeneous geometries, i.e., the contours of which may be superimposed with more than 50% of the portions overlapping or being parallel.

The meshes of each layer may be separated from one another by material strands each having homogeneous thicknesses, i.e. not differing by more than 15% from one another.

The implant may have a volume within the range of 0.05 mL to 3 L, preferably 100 mL to 600 mL.

The implant may be a breast implant.

According to another aspect, the present invention relates to the use of a solution comprising a divalent cation, preferably calcium, and transglutaminase, and optionally one or more other divalent cations, to consolidate a hydrogel comprising alginate and gelatin.

This consolidation solution can also be used to consolidate a hydrogel comprising, in addition to alginate and gelatin, fibrinogen and consequently, said solution will comprise thrombin in addition to the divalent cation, preferentially calcium, and transglutaminase, and any other divalent cation(s).

All the above-mentioned preferences relating to the consolidation method apply mutatis mutandis to the use of the consolidation solution according to the invention.

According to another aspect, the present invention relates to a hydrogel comprising consolidated alginate and gelatin, preferably intended to be implanted in a human subject, the hydrogel being obtainable by a consolidation method as described previously.

According to a second embodiment, the present invention relates to a method for consolidating a hydrogel comprising alginate and gelatin, said method comprising, in order, the following steps:
preparation of the hydrogel,
shaping the prepared hydrogel,
contacting the hydrogel with at least one divalent cation, preferably calcium, and transglutaminase, and optionally one or more other divalent cations.

The hydrogel comprising alginate and gelatin may be prepared as described above.

Preferably, the preparation of the hydrogel does not require the shaping of one or more components prior to the preparation of the hydrogel. Preferably, the preparation of the hydrogel does not require the shaping of one or more components, for example alginate and/or gelatin, in the form of fibers prior to the preparation.

Methods for shaping components in the form of fibers are well known by a person skilled in the art and include, for example, electrospinning, extrusion, fragmentation, lyophilization and then fragmentation methods.

Preferably, the preparation of the hydrogel does not require the addition of fibers. Preferably, the hydrogel does not comprise fibers.

Examples of fibers are mentioned above.

The hydrogel can be shaped as described above. Preferably, the shaping of the prepared hydrogel is carried out by extrusion of material, preferably by molding or by additive manufacturing, in particular 3D printing.

Contacting the hydrogel with at least one divalent cation, preferably calcium, and transglutaminase, and optionally one or more other divalent cations, may be carried out concomitantly, using a consolidation solution comprising at least calcium and transglutaminase, and optionally one or more other divalent cations.

Preferably, the consolidation solution and the contact of the hydrogel with said solution are as described above.

Contacting the hydrogel with at least one divalent cation, preferably calcium, and transglutaminase, and optionally one or more other divalent cations, may also be carried out sequentially, i.e. the crosslinking agents are not added at the same time during consolidation.

The hydrogel, once prepared, can be contacted with the solutions as described below, in the following order:
a solution comprising a divalent cation, preferably calcium, and optionally one or more other divalent cations,
a solution comprising transglutaminase.

The hydrogel, once prepared, can be contacted with the solutions as described below, in the following order:
a solution comprising transglutaminase,
a solution comprising a divalent cation, preferably calcium, and optionally one or more other divalent cations.

When the hydrogel contains fibrinogen in addition to alginate and gelatin, the consolidation further comprises crosslinking the fibrinogen with thrombin. This crosslinking can be carried out sequentially with the crosslinking of alginate and gelatin (e.g. before or after the crosslinking of alginate and/or gelatin) or concomitantly.

When the hydrogel contains fibrinogen in addition to alginate and gelatin, the hydrogel, once prepared, can be contacted with the solutions as described below, in the following order:
a solution comprising a divalent cation, preferably calcium, and optionally one or more other divalent cations,
a solution comprising transglutaminase,
a solution comprising thrombin.

When the hydrogel contains fibrinogen in addition to alginate and gelatin, the hydrogel, once prepared, can be contacted with the solutions as described below, in the following order:
a solution comprising a divalent cation, preferably calcium, and optionally one or more other divalent cations,
a solution comprising thrombin,
a solution comprising transglutaminase.

When the hydrogel contains fibrinogen in addition to alginate and gelatin, the hydrogel, once prepared, can be contacted with the solutions as described below, in the following order:
a solution comprising transglutaminase,
a solution comprising a divalent cation, preferably calcium, and optionally one or more other divalent cations,
a solution comprising thrombin.

When the hydrogel contains fibrinogen in addition to alginate and gelatin, the hydrogel, once prepared, can be contacted with the solutions as described below, in the following order:
a solution comprising transglutaminase,
a solution comprising thrombin,
a solution comprising a divalent cation, preferably calcium, and optionally one or more other divalent cations.

When the hydrogel contains fibrinogen in addition to alginate and gelatin, the hydrogel, once prepared, can be contacted with the solutions as described below, in the following order:
a solution comprising transglutaminase,
a solution comprising thrombin, and a divalent cation, preferably calcium,
a solution comprising a divalent cation, preferably calcium.

When the hydrogel contains fibrinogen in addition to alginate and gelatin, the hydrogel, once prepared, can be contacted with the solutions as described below, in the following order:
a solution comprising thrombin,
a solution comprising transglutaminase,
a solution comprising a divalent cation, preferably calcium.

When the hydrogel contains fibrinogen in addition to alginate and gelatin, the hydrogel, once prepared, can be contacted with the solutions as described below, in the following order:
a solution comprising thrombin,
a solution comprising a divalent cation, preferably calcium,
a solution comprising transglutaminase.

When the hydrogel contains fibrinogen in addition to alginate and gelatin, the hydrogel, once prepared, can be contacted with the solutions as described below, in the following order:
a solution comprising thrombin,
a solution comprising transglutaminase, and a divalent cation, preferably calcium.
a solution comprising a divalent cation, preferably calcium.

During consolidation, the contact of the hydrogel with the aforementioned solution(s) can be carried out by immersion, during which the hydrogel is immersed in its entirety in the aforementioned solution(s). It can also be carried out by imbibition, by spraying, using a drip, trickling, or similar system.

According to another aspect, the present invention relates to a hydrogel comprising consolidated alginate and gelatin, preferably intended to be implanted in a human subject, the hydrogel being obtainable by a consolidation method as described previously.

### BRIEF DESCRIPTION OF THE DRAWINGS

Other features, aims and advantages of the invention will emerge from the following description, which is purely illustrative and non-limiting, and which must be read with reference to the appended drawings in which:

FIG. 1 represents the comparison of Young's modulus (A) and viscosity (B) of AG and FAG hydrogels which constitute implants according to the invention.

FIG. 2 represents the comparison of Young's moduli E0 (Pa) of an AG hydrogel in which the gelatin is crosslinked with and without transglutaminase and stored for up to 7 days at 37° C.

FIG. 3 represents the comparison of Young's moduli E0 (Pa) of an AG hydrogel and commercial hydrogels crosslinked or not with transglutaminase. *: Liquid compound at 37° C. +: visible polymerization but stiffness of the gel insufficient at 37° C. for DMA measurement.

FIG. 4 represents the viability and cell growth measured kinetically on FAG and AG hydrogels which constitute implants according to the invention, and which have been colonized in vitro by fibroblasts, after their manufacture.

FIG. 5 represents the viability and cell growth measured kinetically on FAG and AG hydrogels which constitute implants according to the invention, and which have been colonized in vitro by stem cells of adipose tissue, after their manufacture.

FIG. 6 shows the metabolic activity of AG implants according to the invention at different culture points following their in vitro colonization by a fraction of purified adipose tissue, after their manufacture.

FIG. 7 represents histological analyses by Hematoxylin, Phloxine, Safran (HPS) staining of AG implants according to the invention after 2 days (4 left images) or 7 days (2 right images) of in vitro incubation with a fraction of purified adipose tissue, after their manufacture (Top: external edges of the matrices; Bottom: internal pores of the matrices; images taken in white light; magnification 100×; scale 100 μm).

FIG. 8 represents perilipin-1 immunostaining and Dapi staining of cell nuclei, on AG implants according to the invention after 2 days (top image) or 7 days (bottom image) of in vitro incubation with a purified adipose tissue fraction, after their manufacture (fluorescence imaging; magnification 200×; scale 50 μm).

FIG. 9 represents the comparison of the Young's moduli of AG implants for crosslinking with variable durations at 21° C. (B) and 37° C. (A).

FIG. 10 represents the comparison of Young's moduli E0 (A-C) and viscosities (D-F) of AG and FAG implants after crosslinking with different concentrations of CaC12, TAG and thrombin.

FIG. 11 represents the comparison of Young's moduli E0 (A-B) and viscosities (C-D) of AG and FAG implants after sequential or concomitant crosslinking with CaC12, TAG and thrombin.

FIG. 12 represents the comparison of Young's moduli E0 (A) and viscosities (B) of AG and FAG implants after crosslinking with a solution containing calcium chloride or barium chloride.

FIG. 13A illustrates the variation of dimensions (A1-A2) and of pores (A3-A4) of AG and FAG implants according to the invention before and after crosslinking. FIG. 13B illustrates the impact of sterilization on dimensions (B1-B2) and Young's modulus (B3-B4) of these implants.

FIG. 14 illustrates the repeatability of the production of AG implants according to the invention in terms of dimensions (A), volume (B) and porosity (C).

FIG. 15 illustrates the repeatability of the shrinkage of AG implants according to the invention after consolidation.

FIG. 16 illustrates the repeatability of the shrinkage of AG implants according to the invention as a function of the sterilization method.

FIG. 17A illustrates the repeatability of the extrusion diameter. FIG. 17B illustrates the length of the pores (B1-B2) of AG implants according to the invention.

FIG. 18 represents images of pores of variable size in an AG implant according to the invention.

FIG. 19 represents the surgical plan (on the left) for the in vivo subcutaneous implantation (on the right) of AG and FAG implants according to the invention.

FIG. 20 shows histological analyses after staining with Masson trichrome on sections of AG implants according to the invention, after subcutaneous in vivo implantation in rat back sites for 3 weeks (images at low, medium and high magnification).

FIG. 21A shows the analysis over 28 days of cell survival measured by lactate accumulation.

FIG. 21B represents the analysis over 28 days of cell growth followed by calcein labeling in a cellularized FAG hydrogel.

FIG. 22 represents the Young's moduli (E0) measurement as a function of the initial cell concentration in a cellularized FAG hydrogel.

FIG. 23 represents the evolution over 21 days of the lactate concentration measured in culture supernatants from cellularized and consolidated AG or FAG hydrogels.

FIG. 24 represents histological analyses after HPS staining of bioprinted, full (left panel) or porous (right panel), FAG fibroblasts/endothelial cells hydrogel constructs after 21 days of culture.

FIG. 25 represents histological analyses after HPS staining of bioprinted, full (left panel) or porous (right panel), AG fibroblasts/endothelial cells hydrogel constructs after 21 days of culture.

FIG. 26 represents CD31-DAB immunolabeling (black) on full bioprinted, FAG fibroblasts/endothelial cells hydrogel constructs after 21 days of culture.

FIG. 27 represents CD31-DAB (black) immunolabeling on porous bioprinted, FAG fibroblasts/endothelial cells hydrogel constructs after 21 days of culture.

FIG. 28 represents histological analysis after HPS staining of skins reconstructed from 3D bioprinted FAG hydrogel and consolidated with TAG.

### EXAMPLES

The present invention will be better understood on reading the following examples which illustrate the invention in a non-limiting manner.

### Materials and Methods:

Protocol #1 Preparation of an AG hydrogel: In order to prepare the AG hydrogel, 2 g of alginate (very low viscosity, Alpha Aesar, France), 5 g of gelatin (Sigma-Aldrich, France) are dissolved at 37° C. for 12 H in 100 mL of a 0.1M NaCl solution (Labelians, France).

Protocol #2 Preparation of a FAG hydrogel: In order to prepare the FAG hydrogel, 2 g of alginate (very low viscosity, Alpha Aesar, France), 5 g of gelatin (Sigma-Aldrich, France) and 2 g of fibrinogen (Sigma-Aldrich, France) are dissolved at 37° C. for 12 H in 100 ml of a 0.1M NaCl solution (Labelling, France).

Protocol #3 Molding of an AG or FAG hydrogel: 1.8 mL of the hydrogel prepared according to protocol #1 or #2 are deposited in the wells of a 6-well culture plate and incubated at 21° C. for 30 minutes.

Protocol #4 Crosslinking of an AG hydrogel: a crosslinking solution is prepared by dissolving 4 g of transglutaminase (Ajinomoto, Japan), 3 g of CaC12 (Sigma Aldrich, France) in 100 mL of a 0.1M NaCl solution (Labeling, France). The crosslinking solution is then brought into contact with the hydrogel for 1H30 at 37° C. (unless otherwise indicated).

Protocol #5 Crosslinking of a FAG hydrogel: A crosslinking solution is prepared by dissolving 4 g of transglutaminase (Ajinomoto, Japan), 3 g of CaC12 (Sigma Aldrich, France) and 400 units of thrombin (Sigma Aldrich, France) in 100 mL of a 0.1M NaCl solution. The crosslinking solution is then brought into contact with the hydrogel for 1H30 at 37° C. (unless otherwise indicated).

Protocol #6 Dynamic Mechanical Analysis (DMA) in compression: The mechanical properties of the FAG and AG hydrogels are measured in triplicate with a rotational rheometer (DHR2, TA Instrument, France), a Peltier plane (TA Instrument, France) and a 8 mm notched geometry (TA Instrument, France). Three 8 mm diameter disks are cut from the molded hydrogels according to protocol #3. The disk is placed on the lower geometry at 37° C. for 60 seconds and then a 10 μm oscillatory compression procedure is performed from 0.1 to 10 Hz at 100 μm/s and at 37° C. The values of Young's modulus E0 (Pa) and viscosity η0 (Pa·s) of the hydrogel are obtained from a visco-hyperelastic solid modeling using the E′ and E″ values acquired during the test.

Protocol #7 Preparation of a cellularized hydrogel: In order to prepare the cellularized FAG hydrogel, 0.12 g of alginate (very low viscosity, Alpha Aesar, France), 0.3 g of gelatin (Sigma-Aldrich, France) are dissolved in 6 mL of DMEM culture medium (Gibco Cell culture, Invitrogen, France). Freshly trypsinized cells are put in suspension in 2 mL of a 8% fibrinogen solution (Sigma-Aldrich, France). This cell suspension is then added to the preceding solution in order to form the cellularized FAG. In order to prepare the cellularized AG hydrogel, 0.12 g of alginate (very low viscosity, Alpha Aesar, France), 0.3 g of gelatin (Sigma-Aldrich, France) are dissolved in 6 mL of DMEM culture medium (Gibco Cell culture, Invitrogen, France). Freshly trypsinated cells are put in suspension in 2 mL of a 0.1M NaCl solution (Fabelians, France). This cell suspension is then added to the preceding solution in order to form the cellularized AG.

Protocol #8 3D printing of hydrogels: Hydrogels prepared according to protocol #1, #2 are transferred into a 30 mL cartridge (Nordson EFD) equipped with a 410 μm diameter extrusion nozzle (Nordson EFD). The cartridge-nozzle assembly is then placed in a 3D printer (BioassemblyBot, Advanced Solution Lifescience, USA) allowing constant pressure to be applied to the cartridge while moving in all three directions of space. The printing parameters are a speed of 10 mm/sec, a pressure of 25-35 PSI and a temperature of 21° C. The different filling rates are obtained by the internal slicer of the printer control software (Tsim, Advanced Solution Lifescience, USA).

Protocol #9 In vivo implantation in rats: The in vivo implantation studies in rats were conducted on the BIOVIVO-Institut Claude Bourgelat (Lyon, France) preclinical research technical platform. The experiments were conducted in accordance with the European Directives 2010/63/EU. The 16 animals (Sprague Dawley rat, 250-300 g) were anesthetized by inhalation (oxygen and 5% isoflurane). The dorsal implantation sites were shaved and disinfected with povidone and sterile gauze, and sterile drapes were placed to delineate the surgical area. General anesthesia was maintained with isoflurane (2%) and oxygen inhalation. Pre-surgical analgesia was performed subcutaneously with meloxicam and morphine at 1 mg/kg respectively. Body temperature and pulse rate of the rats were monitored during surgery. Two skin incisions of 2-3 cm were made in the back region. A bioprosthesis was implanted in the dorsal subcutaneous region of each animal. The control group was performed with only the incision and dissection. In one animal per group, 4 surgical sites were performed, three bioprostheses and one control specimen. The surgical site was closed in layers using subcutaneous and cutaneous sutures with absorbable braided sutures (PDS® polidioxanone, 4/0 and Nylon 3/0, Ethicon J&J). Postoperatively, the animals were monitored for signs of suffering, and the surgical wounds were inspected daily for skin healing and absence of infection. Explantation took place 21 days after implantation.

Protocol #10 Histological analysis: Implants were fixed for 24 hours in a 4% formalin solution (Alphapat, France) and then dehydrated by successive baths of absolute ethanol (vwr chemicals, France) and methylcyclohexane (vwr chemicals, France) with an STP 120 dehydrator (Myr, Spain) and then embedded in kerosene (Sakura, Japan). Sections of 5 μm thickness were made with a HM 340e microtome (Microm, France). Hematoxylin Phloxine Saffron (HPS), Masson's Trichrome and DAPI staining were performed.

### Example 1—Mechanical Properties of Alginate/Gelatin (AG) and Fibrinogen/Alginate/Gelatin (FAG) Hydrogels

AG and FAG hydrogels were prepared from protocols #1 and #2, molded according to protocol #3, and then crosslinked using protocols #4 and #5 to study their DMA mechanical properties using protocol #6.

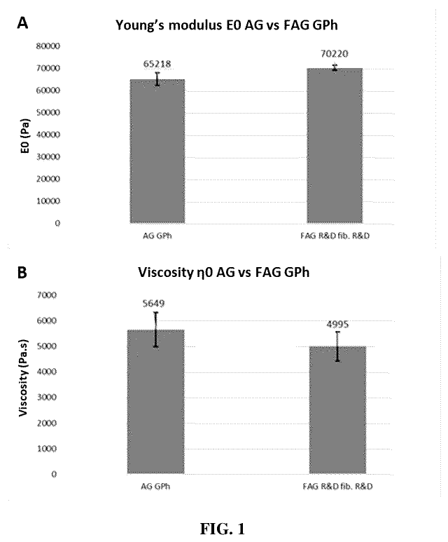

The results are shown in FIG. 1 (A-B). The measured Young's modulus and viscosity values are similar between the AG hydrogel and the FAG hydrogel following their crosslinking by the process of the invention. The Young's moduli under the specific conditions of this study are around 68,000 Pa.

### Example 2—Impact of Crosslinking with Transglutaminase on the Mechanical Properties of Alginate/Gelatin Hydrogel (AG)

Molded samples of AG were prepared from protocols #1 and #3 and crosslinked from a variant of protocol #4. In this variant, the crosslinking solution is composed of a 30 mg/mL calcium chloride solution only or a 30 mg/mL calcium chloride and 40 mg/mL transglutaminase solution. Four gels of each condition were cast and tested in DMA on the same day and after 1, 4 and 7 days of storage at 37° C., respectively, in order to mimic physiological conditions.

The samples were then studied by DMA using protocol #6.

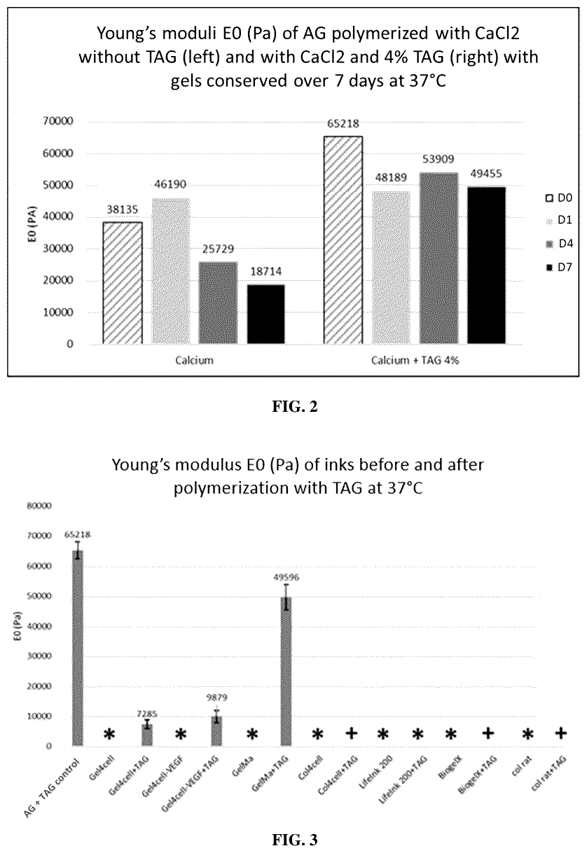

The results are shown in FIG. 2. This study shows the advantageous effect of the use of transglutaminase during crosslinking on the mechanical properties of hydrogels. This effect is even greater when the gels are converted at 37° C., justifying the particular interest of the crosslinking according to the invention for hydrogels intended to be implanted.

### Example 3—Impact of Crosslinking with Transglutaminase on the Mechanical Properties of Commercial Gelatin and/or Collagen Hydrogels

Molded samples of AG were prepared from protocols #1 and #3 and crosslinked from protocol #4. The commercial hydrogel samples listed in Table 1 below were prepared according to the protocols provided by the suppliers and molded according to protocol #3.

TABLE 1

| Hydrogel commercial name | Reference | Supplier |
|---|---|---|
| Gel4Cell | BIS-101 | Bioink Solution |
| Gel4Cell-VEGF | BIS-103 | Bioink Solution |
| Col4Cell | BIS-108 | Bioink Solution |
| GelMA | VL5000000010 | Cellink |
| Lifeink 200 Type I Collagen | 5278-5ML | CellSystems |
| BiogelX | B5X-0000 | Biogelx |
| Rat tail Collagen | A10483-01 | Sigma |

Hydrogels were crosslinked with a variation of protocol #4, using either a solution comprising only calcium at 30 mg/mL (no TAG), or a solution of calcium at 30 mg/mL and transglutaminase at 40 mg/mL, in order to observe the impact of TAG.

The un-crosslinked and crosslinked samples with TAG were then studied by DMA using protocol #6.

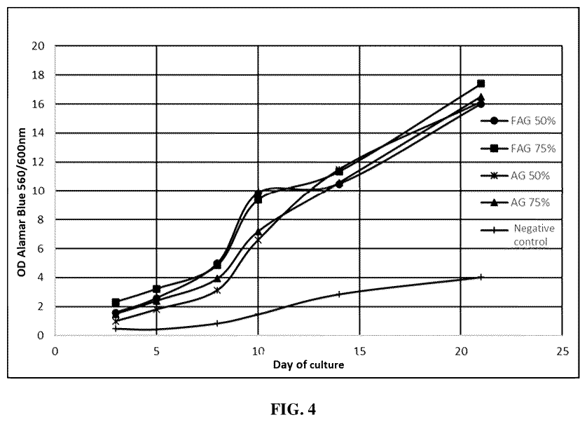

The results are grouped in FIG. 3. Six of the seven commercial hydrogels studied were crosslinked with transglutaminase. Collagen-based hydrogels (Col4Cell, Rat Collagen) are not stiff enough to be analyzed by DMA but gelatin-based hydrogels (Gel4cell, Gel4cell-VEGF and GelMa) have a significantly higher Young's modulus after transglutaminase crosslinking (7.3, 9.9 and 50 kPa respectively). This study shows the effect of crosslinking with transglutaminase on the stiffness of commercial hydrogels.

### Example 4—Influence of the Amount of Alginate and Gelatin in a Fibrinogen/Alginate/Gelatin (FAG) Hydrogel on Mechanical Properties

FAG hydrogels were prepared from a variant of protocol #2, molded according to protocol #3, then crosslinked thanks to protocol #5, then their mechanical properties were studied by DMA thanks to protocol #6. In this variant, we studied these mechanical properties by preparing the FAG hydrogel, with 1 or 3 or 2 g of alginate, and 10 or 7.5 or 5 g of gelatin, respectively, and 2 g of fibrinogen.

The results are grouped in Table 2 below. The Young's moduli under the specific conditions of this study range from 200 to 800 kPa.

TABLE 2

| | | | Young's modulus |
|---|---|---|---|
| Gelatin (%) | Alginate (%) | Fibrinogen (%) | (kPa) |
| 10 | 1 | 2 | 200 |
| 7.5 | 3 | 2 | 600 |
| 5 | 2 | 2 | 800 |

### Example 5—Evaluation of Fibrinogen/Alginate/Gelatin (FAG) and Alginate/Gelatin (AG) Hydrogels Colonization by Fibroblasts

AG and FAG hydrogels were prepared from protocols #1 and #2. Square implants of 1.5 cm side and 0.2 cm thickness were then printed using protocol #8 and crosslinked using protocols #4 or #5. The printed implants were produced with a 50% fill rate and an extrusion nozzle of 410 μm internal diameter. A negative control (empty well) is also used.

Normal human fibroblasts in passage 6 are thawed and amplified in 175 cm2 culture flasks in culture medium containing DMEM supplemented with 10% calf serum and 1% antibiotics. Each implant was seeded on its surface with a cell suspension of normal human fibroblasts at a concentration of 4,000,000 fibroblasts/ml. 250 μl of this suspension was drip-fed onto each implant, i.e. 1,000,000 fibroblasts/implant. After 1 hour of adhesion, the implants were immersed with culture medium. Implants were cultured in culture medium composed of DMEM containing 10% calf serum supplemented with vitamin C and EGF (Epidermal Growth Factor) at 37° C., 5% CO2. Implants were cultured with this same medium for 21 days, renewed every 3 days.

The metabolic activity of the fibroblasts within the implants was studied by colorimetric analysis with Alamar Blue on days 3, 5, 8, 10, 14 and 21 after seeding. The solution was made by diluting 10-fold a solution of Alamar Blue (DAL 1100, Invitrogen) in DMEM. After 19 hours of incubation at 37° C., 100 μl of the supernatants were collected and their absorbance at 570 nm and 600 nm was measured by spectrophotometer (NanoQuant® infinite M200PRO, TECAN).

Cell viability and growth were monitored over 21 days of culture using 6-point kinetics on days 3, 5, 8, 10, 14 and 21. The results are shown in FIG. 4.

The results confirmed that all implants allowed fibroblast adhesion and survival as early as day 3 of culture. Cell growth is observable for each porous implant over the 21 days of culture, on both types of hydrogels (FAG and AG) as well as for each overall porosity employed.

### Example 6: Evaluation of Colonization of Fibrinogen/Alginate/Gelatin (FAG) and Alginate/Gelatin (AG) Hydrogels by Adipose Tissue Stem Cells (ASC)

AG and FAG hydrogels were prepared from protocols #1 and #2. Square implants of 1.5 cm side and 0.2 cm thickness were then printed using protocol #8 and crosslinked using protocols #4 or #5. The printed implants were produced with a 50% and 75% fill rate and an extrusion nozzle of 410 μm internal diameter. Sterilization was performed by the company IONISOS (France) by irradiating the implants with a dose of 30 kGy of Gamma ray.

Normal human adipocyte stem cells in passage 2 to 5 were thawed and amplified in 175 cm2 culture flasks in culture medium containing DMEM supplemented with 10% serum and 1% antibiotics. Each implant was seeded on its surface with a cell suspension of ASC at a concentration of 6, 12, or 24 million ASC/ml. 250 μl of these suspensions were drip-fed onto each implant, i.e. 1.5, 3 or 6 million ASC/implant. After 1 hour of adhesion, the implants were immersed with culture medium. Implants were cultured in culture medium containing DMEM supplemented with 10% serum and 1% antibiotics for 7 days and then in medium containing DMEM supplemented with 10% serum, insulin, rosiglitasone and 1% antibiotics for 14 days. The culture media are renewed every 3 days.

The metabolic activity of fibroblasts within the implants was studied by colorimetric analysis with Alamar Blue on culture days 3, 5, 7, 14 and 21 after seeding. The solution was made by diluting 10-fold a solution of Alamar Blue (DAL 1100, Invitrogen) in DMEM. After 5 hours of incubation at 37° C., 100 μl of the supernatants were collected and their absorbance at 570 nm and 600 nm was measured by spectrophotometer (NanoQuant® infinite M200PRO, TECAN).

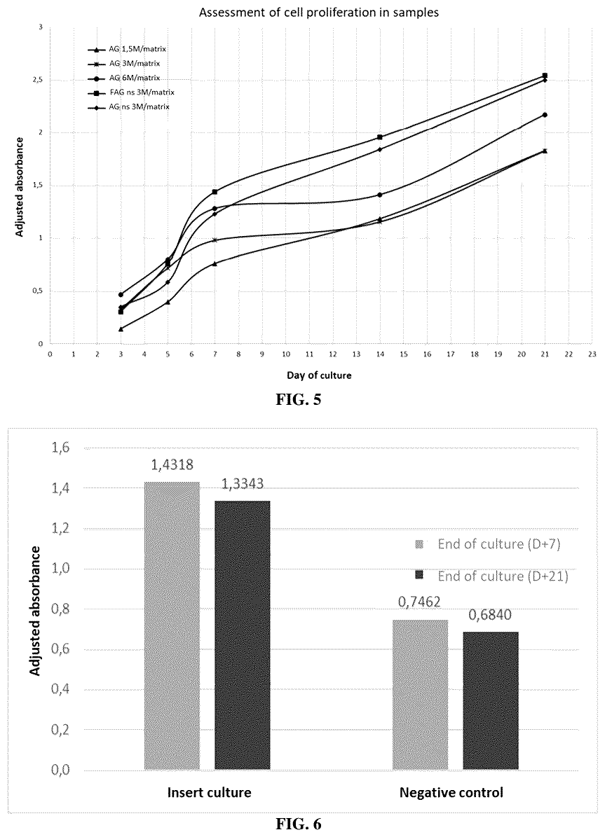

Cell viability and growth were monitored over 21 days of culture using 6-point kinetics on days 3, 5, 7, 14 and 21. The results are shown in FIG. 5.

The results confirmed that all implants allowed adipocyte stem cells to adhere and survive from day 3 of culture. Cell growth is observable for each porous implant over the 21 days of culture, on both types of hydrogels (FAG and AG) as well as for each density of seeding.

### Example 7: Evaluation of the Colonization of Alginate/Gelatin (GA) Hydrogels in Contact with a Purified Adipose Tissue Fraction

AG hydrogels were prepared from protocol #1. Cubic implants of 1.5 cm side and 0.8 cm thickness were then printed using protocol #8 and crosslinked using protocol #4. The printed implants were produced with a 50% fill rate and an extrusion nozzle of 410 μm internal diameter.

The lipoaspirate is centrifuged at 1500 RPM for 2 minutes and then rinsed with 1×PBS. The lipoaspirate was again centrifuged at 1500 RPM for 30 seconds and then the 1×PBS was removed. The lipoaspirate was considered purified.

Each implant was then immersed in 6 mL of purified lipoaspirate, and the whole set was placed in a culture insert in a 6-well plate with incubation in medium containing DMEM supplemented with 10% serum and 1% antibiotics at 37° C. 5% CO2 for 2 days or 7 days.

Following contact with lipoaspirate, the implants were grown in 6-well plates in culture medium containing DMEM supplemented with 10% serum, insulin, rosiglitasone, and 1% antibiotics, with 3 medium changes per week until 21 days.

Cellular metabolic activity within the implants was studied by colorimetric analysis with Alamar Blue on culture days 2, 7, and 21 after seeding. The solution was made by diluting 10-fold a solution of Alamar Blue (DAL 1100, Invitrogen) in DMEM. After 5 hours of incubation at 37° C., 100 μl of the supernatants were collected and their absorbance at 570 nm and 600 nm was measured with a spectrophotometer (NanoQuant® infinite M200PRO, TECAN).

Cell viability and growth were monitored over 21 days. The results are shown in FIG. 6.

A much higher metabolic activity than the negative control was observed in the implants that were in contact with purified lipoaspirate.

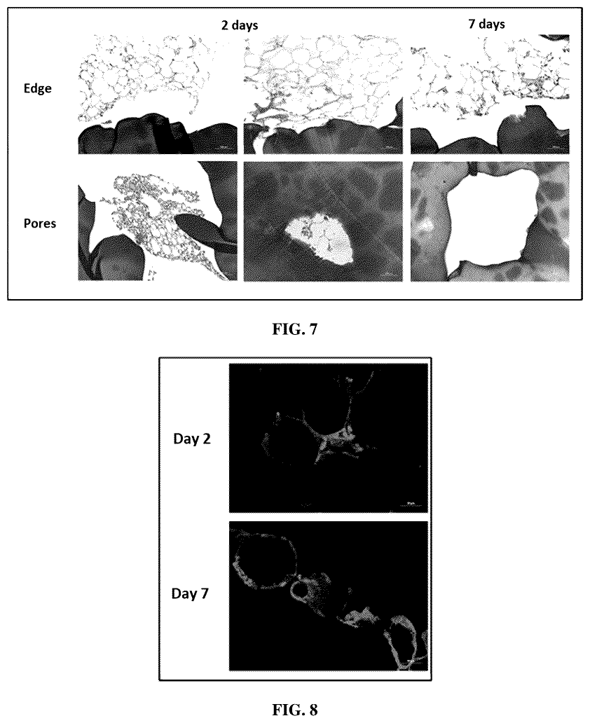

Histological analyses were performed to complete this study according to protocol #10. The results are shown in FIG. 7.

The images reveal the presence of agglomerated, polygonal, uniform, unilocular, and bulky adipocytes. These morphological characteristics are those of healthy adipocytes, which can be found in adipose tissue.

Immunostaining for perilipin-1 was also performed. Samples were included in OCT (CellPath, KMA-0100-00A) and then stored at −80° C. Sections of 16 μm thickness were made for each sample with a cryostat (Microm, HM 520). The sections were then fixed in acetone/methanol (v/v) solution for 20 minutes and rinsed 3 times in 1×PBS. A 1-hour incubation at room temperature in 4% PBS-BSA solution was performed to saturate the aspecific sites. The sections were then incubated overnight at room temperature with perilipin-1-specific primary antibody solution. The next day, the sections were rinsed three times with 1×PBS and then incubated 45 minutes with Alexa fluor 568-coupled secondary antibody solution at room temperature. Then the sections were rinsed three times with 1×PBS and mounted between slide and coverslip with Dapi fluoromount-G® mounting medium (SouthernBiotech). The images obtained are grouped together FIG. 8.

The images show adipocytes with large spherical or polygonal vacuoles depending on the clustering of the cells. The adipocytes appear as unilocular and their size is also physiological as it ranges from 50 to 200 μm.

Taken together, these results confirm the adhesion, survival, and regeneration of a human adipose tissue brought into contact with the implants. The particular structure and composition of the implants thus form a favorable environment for the regeneration of healthy adipose tissue.

### Example 8—Influence of Temperature and Crosslinking Time on the Mechanical Properties of an Alginate/Gelatin Hydrogel (AG)

Molded samples of AG were prepared from protocols #1 and protocol #3 and crosslinked from a variant of protocol #4. In this variation, the crosslinking times and temperatures were changed from 10 minutes to 14H and from 37° C. to 21° C.

The samples were then studied by DMA using protocol #6.

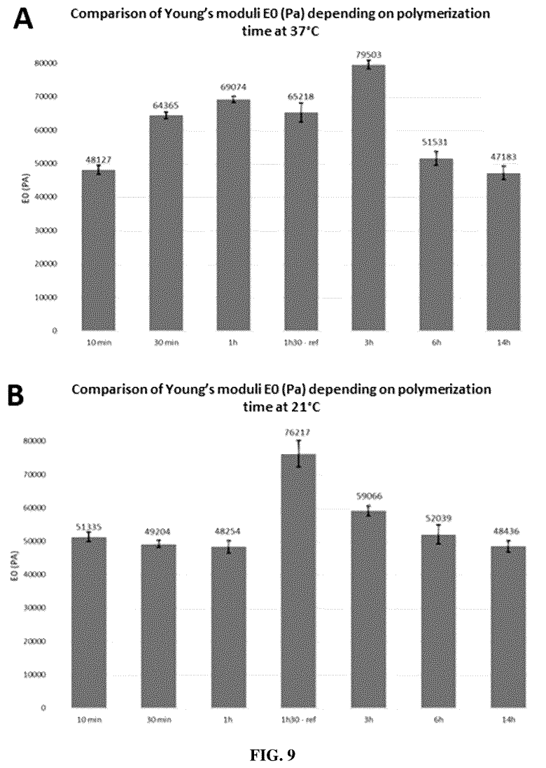

The results are shown in FIG. 9 (A-B). The crosslinking times as well as the crosslinking temperature have very little influence on the final mechanics (Young's modulus) of the hydrogels. However, it seems that an optimum can be found around 1 h30, whatever the temperature.

These Young's moduli are also very stable over 7 days after crosslinking at 37° C. The crosslinking of the gelatin was efficient since there was no loss of dissolved gelatin in the medium.

### Example 9—Impact of Crosslinking Solution Component Concentration on the Mechanical Properties of Alginate/Gelatin (AG) and Fibrinogen/Alginate/Gelatin (FAG) Hydrogels Once Crosslinked

Molded samples of AG and FAG were prepared from protocols #1, #2 and #3, crosslinked from a variant of protocols #4 and #5. In this variation, the concentrations of the components of the crosslinking solution were changed (transglutaminase, calcium chloride, and thrombin).

The samples were then studied by DMA using protocol #6.

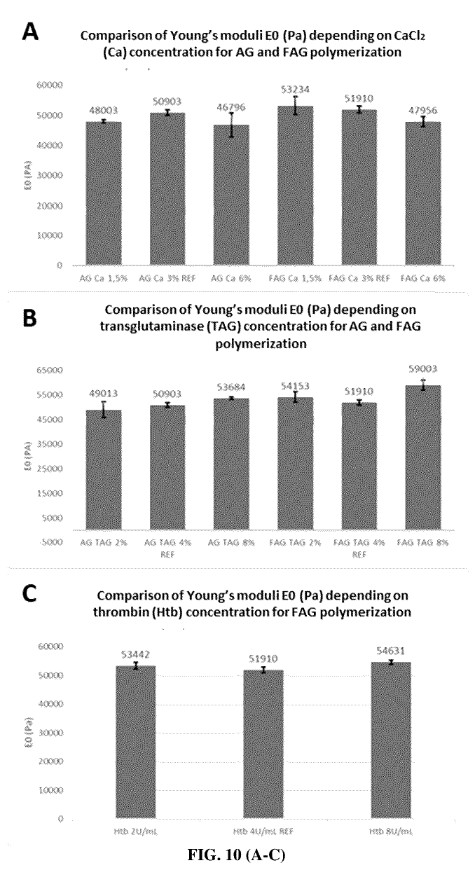

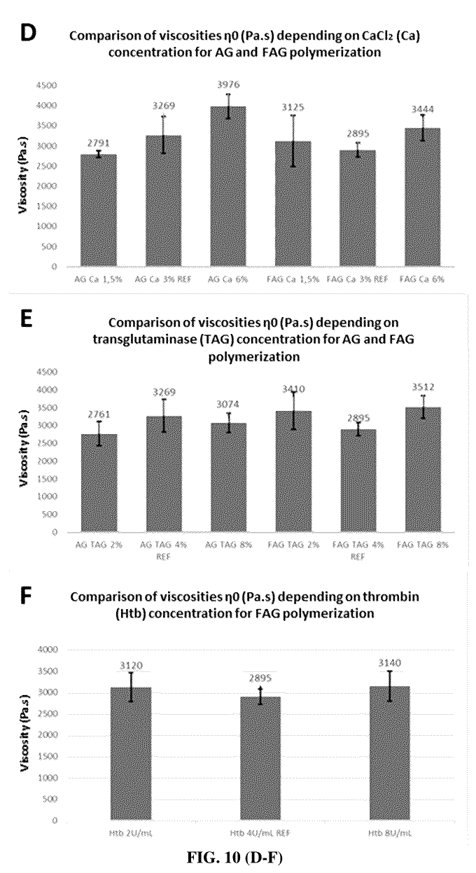

The results are grouped in FIG. 10 (A-F). Over this range of reagent concentrations, no significant variations were observed (E0 all very similar).

### Example 10—Impact of Sequential or Concurrent Crosslinking of Alginate/Gelatin (AG) and Fibrinogen/Alginate/Gelatin (FAG) Hydrogel

Molded samples of AG and FAG were prepared from protocols #1, #2, and #3, crosslinked from a variant of protocols #4 and #5. In this variant, we investigated sequential crosslinking on FAG and AG, which involves crosslinking the hydrogel in several steps. Each step took 1 h and three rinses with 0.1M NaCl solution were performed between each step to remove residual crosslinking agents. The conditions tested for sequential crosslinking are described in the table below (each step consists in immersing the hydrogel in the solution described for 1 h):

| Type of | | | |
|---|---|---|---|
| hydro- | Step 1 of the | Step 2 of the | Step 3 of the |
| gel | crosslinking | crosslinking | crosslinking |
| AG | Solution of | Solution of | / |
| | 40 mg/mL | 30 mg/mL CaCl2 | |
| | transglutaminase | | |
| AG | Solution of | Solution of | / |
| | 30 mg/mL CaCl2 | 40 mg/mL | |
| | | transglutaminase | |
| AG | Solution of | Solution of | / |
| | 15 mg/mL CaCl2 | 40 mg/mL | |
| | | transglutaminase | |
| AG | Solution of | Solution of | / |
| | 15 mg/mL CaCl2 | 40 mg/mL | |
| | | translutaminase | |
| | | and of 30 mg/mL | |
| | | CaCl2 | |
| AG | Solution of | / | / |
| | 40 mg/mL | | |
| | translutaminase | | |
| | and of 30 mg/mL | | |
| | CaCl2 | | |
| FAG | Solution of | Solution of 4 U/mL | Solution of |
| | 30 mg/mL CaCl2 | thrombin | 40 mg/mL |
| | | | transglutaminase |
| FAG | Solution of | Solution of | Solution of 4 U/mL |
| | 30 mg/mL CaCl2 | 40 mg/mL | thrombin |
| | | transglutaminase | |
| FAG | Solution of 4 U/mL | Solution of | Solution of |
| | thrombin | 30 mg/mL CaCl2 | 40 mg/mL |
| | | | transglutaminase |
| FAG | Solution of 4 U/mL | Solution of | Solution of |
| | thrombin | 40 mg/mL | 30 mg/mL CaCl2 |
| | | transglutaminase | |
| FAG | Solution of | Solution of 4 U/mL | Solution of |
| | 40 mg/mL | thrombin | 30 mg/mL CaCl2 |
| | transglutaminase | | |
| FAG | Solution of | Solution of | Solution of 4 U/mL |
| | 40 mg/mL | 30 mg/mL CaCl2 | thrombin |
| | transglutaminase | | |
| FAG | Solution of | Solution of 4 U/mL | / |
| | 40 mg/mL | thrombin and of | |
| | transglutaminase | 30 mg/mL CaCl2 | |
| | and of 30 mg/mL | | |
| | CaCl2 | | |
| FAG | Solution of 4 U/mL | Solution of | / |
| | thrombin and of | 40 mg/mL | |
| | 30 mg/mL CaCl2 | transglutaminase | |
| | | and of 30 mg/mL | |
| | | CaCl2 | |
| FAG | Solution of | / | / |
| | 40 mg/mL | | |
| | transglutaminase, | | |
| | of 30 mg/mL CaCl2 | | |
| | and of 4 U/mL | | |
| | thrombin | | |

The samples were then studied by DMA using protocol #6.

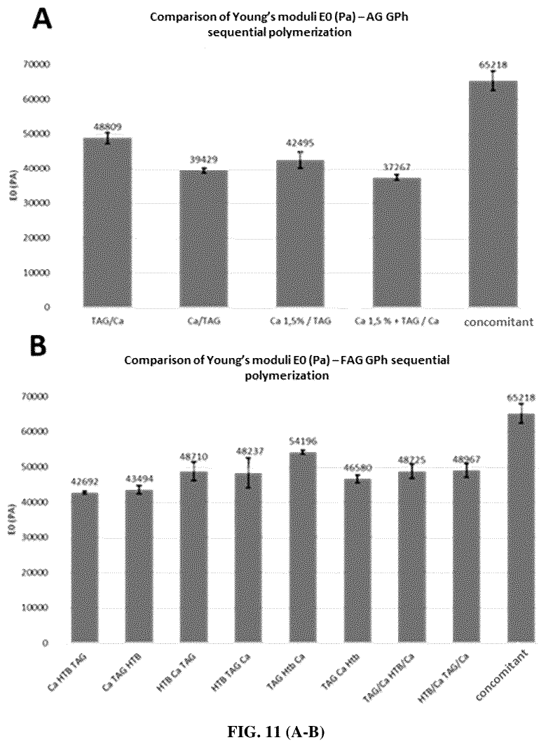

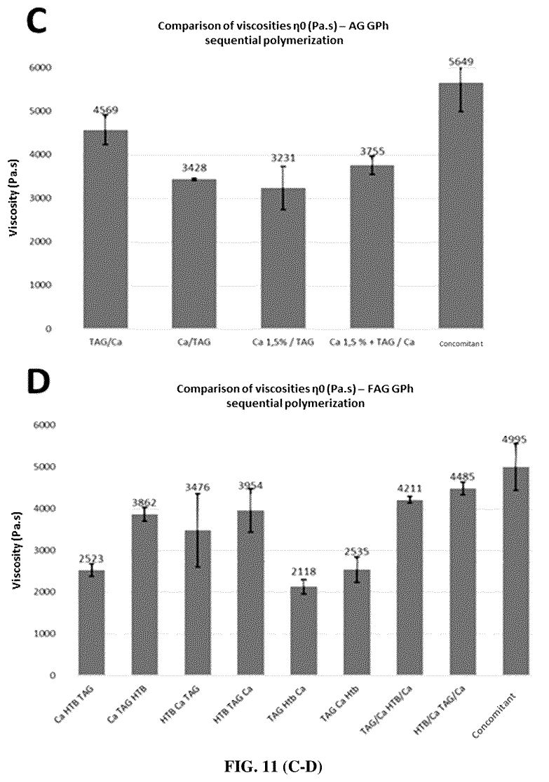

The results are shown in FIG. 11 (A-D). The sequential crosslinking set (FAG and AG) generate hydrogels with lower Young's moduli than the single step crosslinking.

It can be observed that very soft and brittle gels are obtained if calcium is not added first, indeed TAG and thrombin are calcium-dependent, their activity is therefore largely decreased without the addition of CaCl2. The gels are therefore difficult to manipulate without the calcium crosslinking. When thrombin is added first, the gels have very little mechanical strength and holes appear.

### Example 11—Impact of the Nature of the Divalent Cation for the Crosslinking of Alginate/Gelatin (AG) and Fibrinogen/Alginate/Gelatin (FAG) Hydrogels

Molded samples of AG and FAG were prepared from protocols #1, #2, and #3, crosslinked from a variant of protocols #4 and #5. In this variant, we studied a crosslinking in the presence of barium chloride 30 mg/mL.

The samples were then studied by DMA using protocol #6.

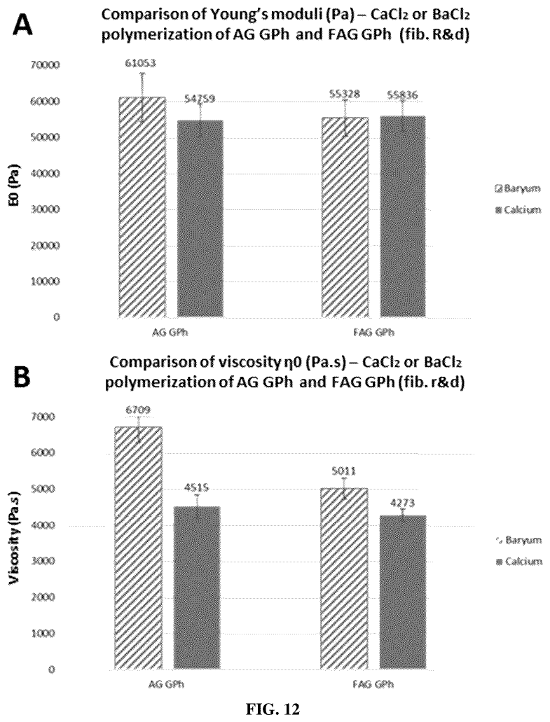

The results are shown in FIG. 12 (A-B). Crosslinking in the presence of barium results in gels with Young's moduli very similar to those obtained with CaCl2. However, since barium increases the viscosity of the gels, the formation of additional pendant chains can be assumed.

### Example 12—Maintenance of the Three-Dimensional Structure and Mechanical Properties of Alginate/Gelatin (AG) and Fibrinogen/Alginate/Gelatin (FAG) Hydrogel Implants after Sterilization

The AG and FAG hydrogels were prepared from protocols #1, #2 and #3, crosslinked using protocols #4 and #5, optically observed and then studied by DMA using protocol #6. The printed shapes are half-spheres of 2 cm diameter produced with variable fill rates (30, 50 and 75%).

Sterilization was performed by the company IONISOS (France) by irradiation of the implants with a variable dose (30 kGy and 40 kGy) of Gamma ray.

The impact of the crosslinking step on the dimensions of alginate/gelatin and fibrinogen/alginate/gelatin hydrogel implants was studied. These dimensions were measured from macroscopic images.

The dimensions of the pores obtained as a function of the fill rate were also studied. These dimensions were measured from images made with a microscope (Olympus, x4 magnification).

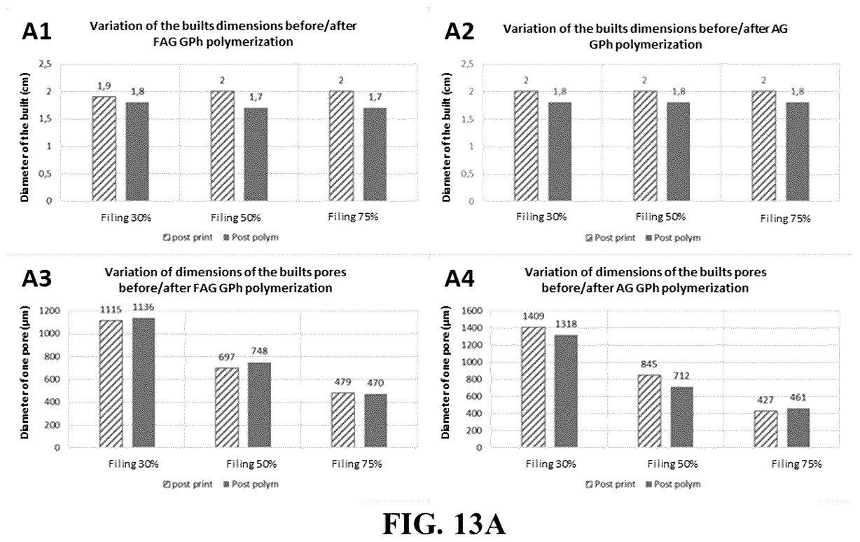

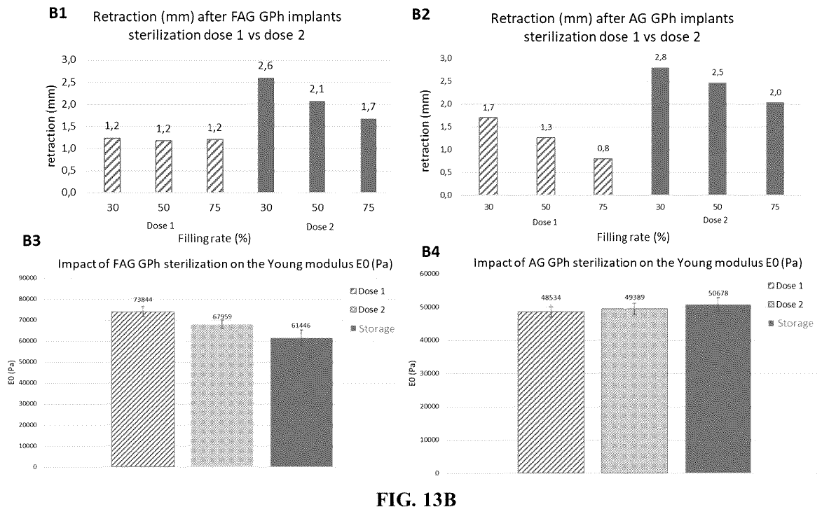

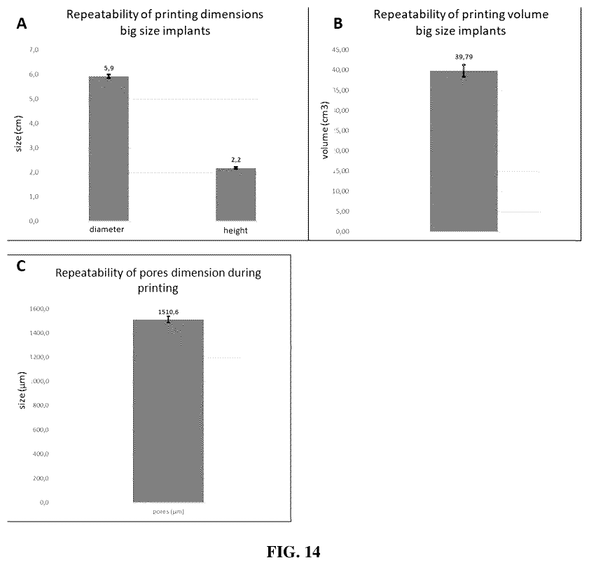

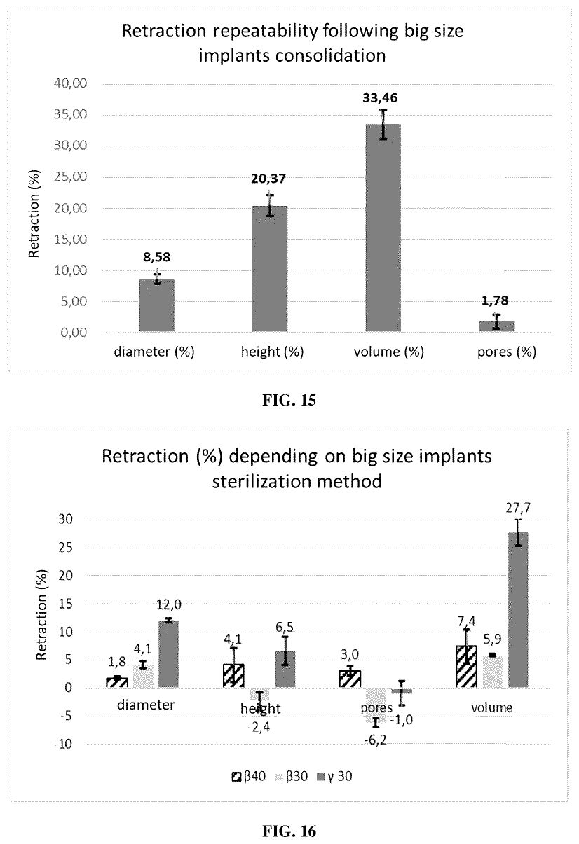

The results are shown in FIG. 13 (A-B). The implants shrink by an average of 10% following the crosslinking step. However, the pore size does not vary significantly (FIG. 13A (A1-A4)).

Regarding sterilization, it appears that the 40 kGy dose leads to a higher shrinkage of the constructs than the 30 kGy dose. Concerning E0, sterilization does not lead to any change in the mechanics of the material for both doses (FIG. 13B (B1-B4)).

### Example 13: Production Quality of Large Alginate/Gelatin (AG) Hydrogel Implant: Repeatability of Impression Dimensions, Dimensions after Consolidation and Sterilization of the Implant by Several Methods

AG hydrogels were prepared from protocol #1. Half-sphere shaped implants of 6 cm diameter and 2 cm thickness were then printed according to protocol #8 and crosslinked using protocol #4, then optically observed and measured. The printed shapes were produced with variable filling rates (25 to 65%) and extrusion nozzles of 410 or 840 μm internal diameter. Sterilization was performed by IONISOS (France) by irradiating the implants with 2 doses (30 kGy and 40 kGy) of Beta rays or a range dose of 30 kGy.

The impact of the crosslinking and sterilization step on the dimensions of large alginate/gelatin hydrogel implants was studied. These dimensions were measured from macroscopic images.

The dimensions of the pores obtained as a function of the filling rate were also studied. These dimensions were measured from images made with a microscope (Olympus, ×4 magnification).

The results after printing are shown in FIG. 14 (A-C). These results show a high repeatability of the dimensions of the large 3D printed implants, reflecting a high production quality.

The results after consolidation of the implants are grouped FIG. 15. This graph shows a high repeatability of the shrinkage of the large implants after the consolidation stage.

The results after sterilization of the implants by 3 methods (β-rays doses 40 and 30 kGy and γ-rays 30 kGy) are grouped FIG. 16. These results show less shrinkage of the large implants with the β 30 and 40 kGy rays.

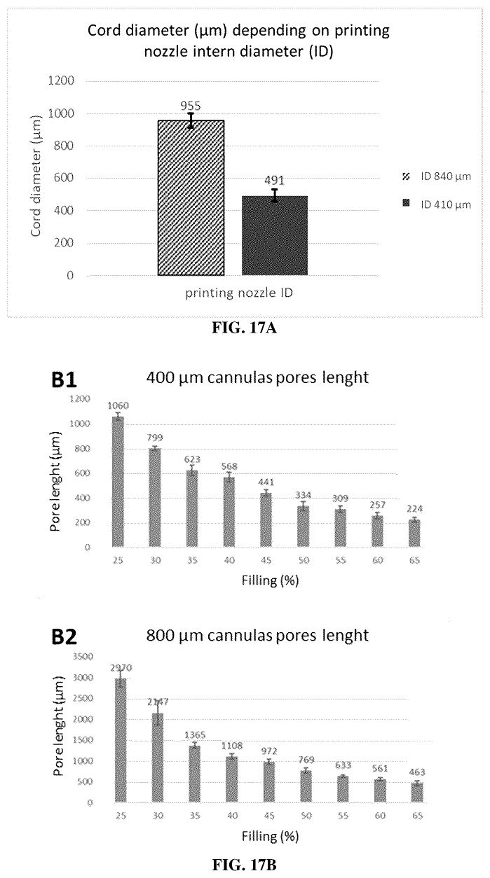

Large implants were printed with 2 extrusion nozzles with internal diameters of 410 and 840 μm with fill rates of 25 to 65%. The repeatability of the extrusion diameter as well as the obtained pore length were measured. The results are shown in FIG. 17 (A-B).

FIG. 17A shows the high repeatability of the extruded bead size. FIG. 17B (B1-B2) shows the variation of pore length with the filling rate of the hydrogel.

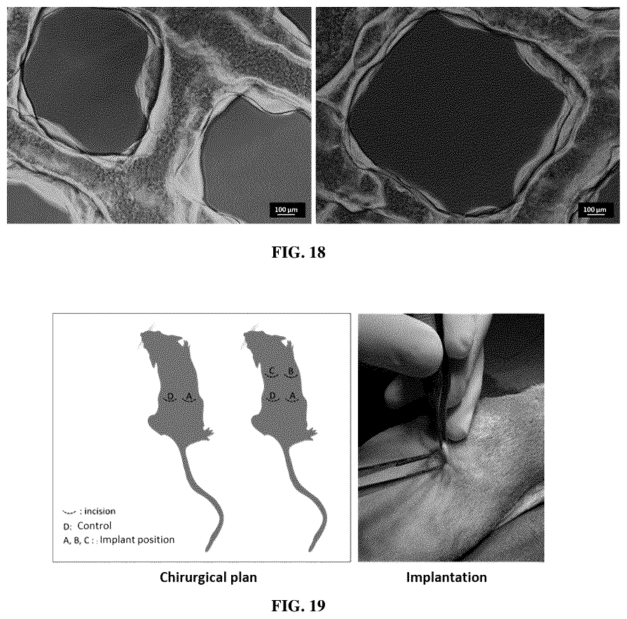

Images of varying pore sizes were taken and are grouped in FIG. 18.

These data show the wide range of pores that can be obtained for the implants and their high repeatability and production quality.

### Example 14—Studies of the In Vivo Resistance of Implants

AG and FAG hydrogels were prepared from protocols #1, #2 and #8, crosslinked through protocols #4 and #5. The printed shapes were 1 cm diameter half-spheres, produced with varying fill rates (30, 50 and 75%).

The porous half-spheres were sterilized with a dose of 30kGy and then implanted subcutaneously in rats according to protocol #9.

Details of the implantation groups are described in the following Table 3, which refers to the surgical implantation plan described in FIG. 19.

TABLE 3

| Group (hydrogel/ | | | position of the |
|---|---|---|---|
| filling rate) | no animal | no implant | implant |
| FAG 30% | 1 | 1 | A |
| | 2 | 2 | A |
| | 3 | 3 | A |
| | 4 | 4 | A |
| | | 5 | B |
| | | 6 | C |
| AG 30% | 5 | 7 | A |
| | 6 | 8 | A |
| | 7 | 9 | A |
| | 8 | 10 | A |
| | | 11 | B |
| | | 12 | C |
| AG 50% | 9 | 9 | A |
| | 10 | 14 | A |
| | 11 | 15 | A |
| | 12 | 16 | A |
| | | 17 | B |
| | | 18 | C |
| AG 75% | 13 | 19 | A |
| | 14 | 20 | A |
| | 15 | 21 | A |
| | 16 | 22 | A |
| | | 23 | B |
| | | 24 | C |

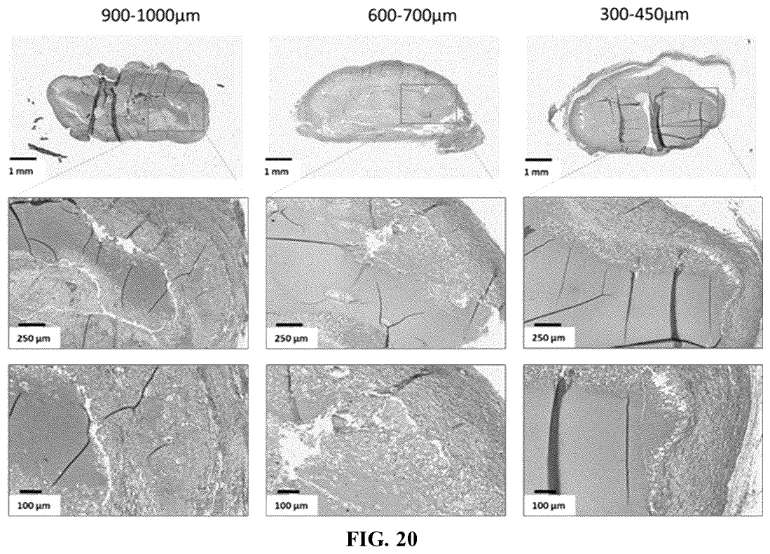

Histological analyses were performed using protocol #10, and the results are grouped in FIG. 20. Explantation was used to validate the resistance of the implants to skin tension. Histological analyses were used to assess cell colonization, vascularization, extracellular matrix synthesis, and the presence of areas of inflammation.

### Example 15—Viability and Cell Growth and Mechanical Properties of Fibrinogen/Alginate/Gelatin Hydrogel (FAG)

Cellularized FAG hydrogels were prepared from the protocol #7 in the presence of different concentrations of human dermal fibroblasts (0.5/0.25/0.125 million cells/mL of hydrogel). Slabs of 1 cm², 0.2 cm thick and 100% filling were prepared using protocol #8 and then crosslinked using protocol #5.

The various slabs were then cultured for 28 days at 37° C. and 5% CO2 in a Dulbecco's Modifier Eagle Medium (DMEM)/Glutamax medium (Gibco Cell Culture, Invitrogen, France), supplemented with 10% (v/v) of bovine calf serum (Gibco Cell Culture, Invitrogen, France), 0.5% (v/v) of amphotericin B (Gibco Cell Culture, Invitrogen, France) and 1% of Penicillin/Streptomycin.

In order to follow cell growth, dosages of the produced L-lactic acid were carried out every two days thanks to a commercial kit (L-Lactic Acid Assay kit, Megazyme) according to the protocol recommended by the supplier. Optical density measurements were carried out with an INFINITE plate reader (TECAN, France).

In order to observe the presence of living cells within the hydrogel slabs along the culture, viability marking was carried out every 5 days in the presence of 1 mM Calcein-AM (Thermofisher, France).

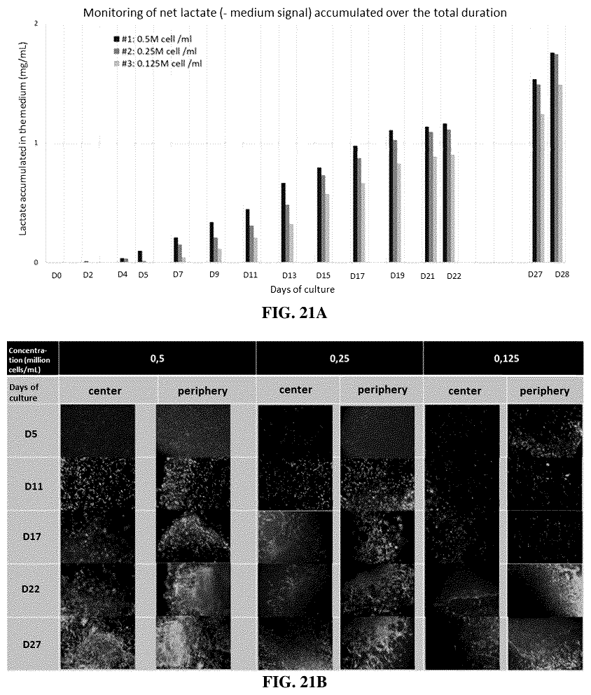

The results are grouped in FIG. 21A. Synthesis of lactate is an indicator of cellular activity. A sharp increase in the amount of lactate is observed from D15 at all cell concentrations, indicating cell growth in the consolidated bioprinted hydrogels according to the invention.

Images of the markings by calcein-AM grouped in FIG. 21B show a viable and increasing cell presence from D11 and a spreading of the cells from D17 for 0.25 M cells/mL and from D22 for the other cell concentrations.

Hydrogels of cellularized FAG prepared and cultured as described above were studied according to protocol #6.

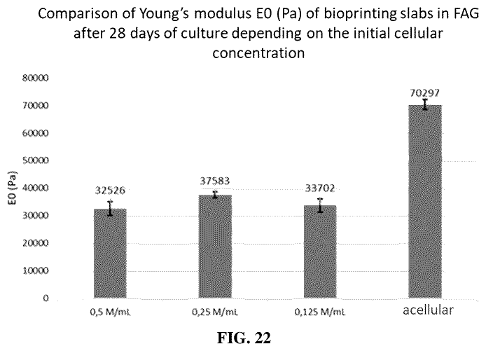

The results are grouped in FIG. 22. The Young's modulus of the hydrogels cellularized after 28 days of culture is not dependent on the cell concentration used during the production. These Young's moduli are however less than those obtained from non-cellularized hydrogels, which denotes a remodeling of the hydrogel by the cells.

### Example 16—Bioprinting of Equivalents Dermis in Consolidated Alginate/Gelatin (AG) and Fibrinogen/Alginate/Gelatin (FAG) Hydrogels

Cellularized AG and FAG hydrogels were prepared from the protocol #7 in presence of a mixture of human dermal fibroblasts (0.25 million cells/mL of hydrogel) and human dermal microvascular endothelial cells (1 million cells/mL of hydrogel). Slabs of 2.25 cm², 0.2 cm thick and filling at 50 and 100% were prepared using protocol #8 and then consolidated using protocols #4 and #5.

The different constructs were then cultured for 21 days at 37° C. and 5% C02 in a culture medium suitable for culturing human dermis composed of DMEM supplemented with 10% of calf serum, 1% of antibiotics, vitamin C and EGF.

In order to follow cell growth, dosages of the produced L-lactic acid were carried out every two days thanks to a commercial kit (L-Lactic Acid Assay kit, Megazyme) according to the protocol recommended by the supplier. Optical density measurements were carried out by means of an INFINITE plate reader (TECAN, France).

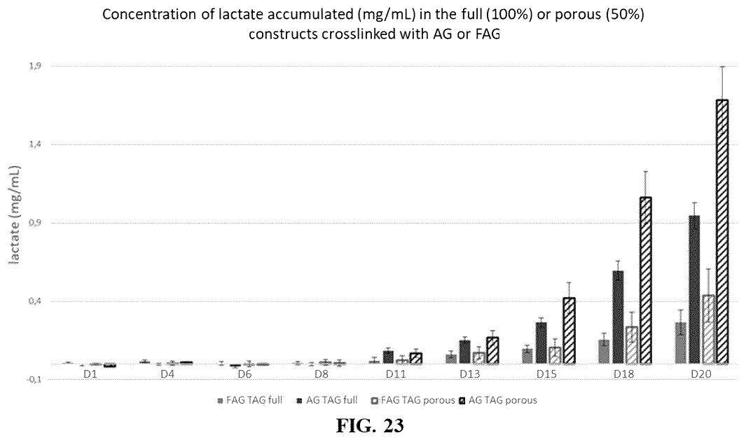

The results are grouped in FIG. 23. An increase in lactate production by cells is observed from D11 under all conditions. The production of lactate is higher in the AG-based hydrogels and in the porous constructs. These results indicate cell proliferation in the hydrogels consolidated for 21 days.

Histological analyses were carried based on the protocol #10. HPS colorations were carried out for all the conditions. In order to evaluate the presence of endothelial cells in the hydrogels, a CD31 immunohistochemical labeling with DAB revelation specific for endothelial cells was carried out on 5 μm thick paraffin cuts.

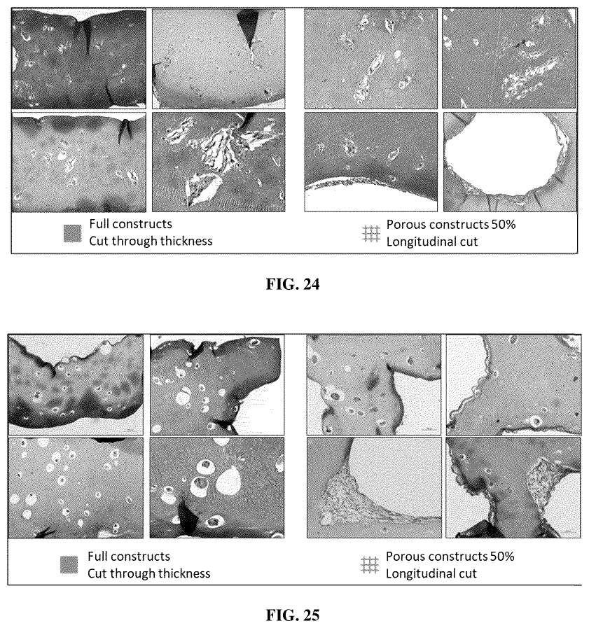

The results are grouped in FIG. 24 to FIG. 27:

FIG. 24: images of the full FAG constructs show that numerous cells are present in the gel and seem to proliferate in the form of agglomerate with adhesion sites on the gel. For porous constructs, clusters of proliferation in the gel are also observed, but also a proliferative layer on the pores surface. We can also observe the beginning of degradation of the hydrogel around the cells in the gel.

FIG. 25: images of the full AG constructs show that numerous cells are present in the gel. For porous constructs, clusters of proliferation are also observed in the gel, but also a surface proliferative layer and areas of high cell density in the corners of the pores. Degradation of the hydrogel around the cells is also observed in the gel.

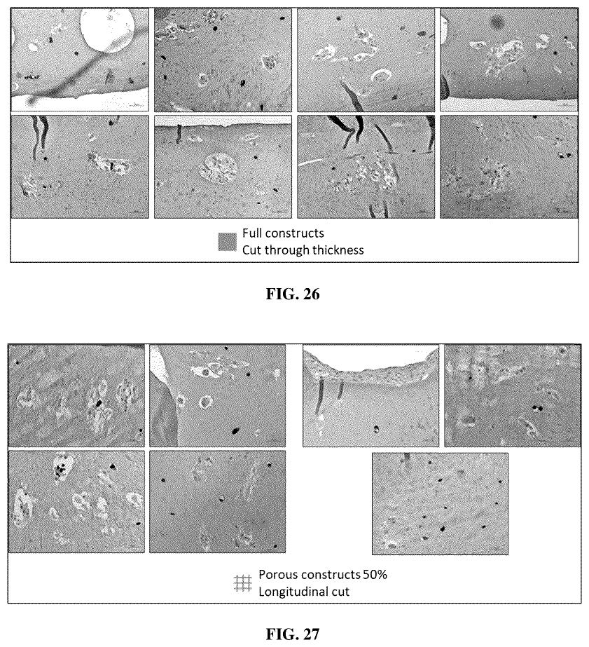

FIG. 26: images of the full FAG constructs reveal the presence of consequent cell clusters of fibroblasts in the gel with small clusters of endothelial cells located around and in the fibroblast clusters.

FIG. 27: images of the porous FAG constructs reveal the presence of cell clusters of fibroblasts in the gel with clusters of endothelial cells located around and in the clusters of fibroblasts. A proliferative layer of fibroblasts is also observed on the surface.

### Example 17—Production of Equivalent Skins by Epidermisation of Bioprinted Products from Consolidated Fibrinogen/Alginate/Gelatin (FAG) Hydrogels

Cellularized FAG hydrogels were prepared from protocol #7 in the presence of a mixture of human dermal fibroblasts. Two distinct conditions were tested: bilayer bioprinting at 1,000,000 fibroblasts/ml and hydrib bilayer bioprinting of an acellular lower layer and a cellularized upper layer with 2,000,000 fibroblasts/ml. For the cellularized bilayer constructs, slabs of 2.2 cm×2.2 cm×0.2 cm (two layers) and 100% filling were printed using the protocol #8 and then consolidated using the protocols #5. For hybrid cellularized bilayer constructs, a first acellular layer of dimensions 2.2 cm×2.2 cm×0.1 cm and then a second cellularized layer (2,000,000 fibroblasts/ml) of dimensions 2.2 cm×2.2 cm×0.1 cm and 100% filling were printed using protocol #8 and then consolidated using protocols #5.

The various constructs were then cultured for 21 days at 37° C. and 5% CO2 in a culture medium suitable for culturing human dermis composed of DMEM supplemented with 10% of calf serum, 1% of antibiotics, vitamin C and EGF. After three weeks of culture, a suspension of normal human keratinocytes at 4,000,000 c/ml was prepared in suitable culture medium, composed of DMEM/HAMF12 supplemented with 10% of calf serum, 1% of antibiotics, insulin, hydrocortisone, vitamin C and EGF. 250 μl of this suspension were deposited on each construct for a seeding of 250,000 keratinocytes/cm². After seeding, the various constructs were cultured for 21 days at 37° C. and 5% CO2 in culture media suitable for culturing equivalent human skins, composed of DMEM/HAMF12 supplemented with insulin, hydrocortisone, vitamin C and 1% antibiotics.

The collagen content of the bioprinted constructs was evaluated under the various conditions. The quantitative analysis of the collagen present in the supports was carried out using a Sircol test (Kit S1000, Biocolor). The control for this study was an acellular FAG construct produced according to the same methodology as the bioprinted and surfaced samples. The pellets resulting from the degradation of collagen were dissolved in 250 μl of basic reagent solution (kit). Absorbance was then measured at 555 nm ((NanoQuant® infinite M200PRO, TECAN) and the results compared with those of a standard range to enable assessment of collagen concentration in digestion supernatants. This concentration was then added to the mass of each sample in order to evaluate their collagen concentration.

The results are grouped in Table 4 below, in which the neosynthesized collagen concentration is indicated for each type of construct. These results were obtained by subtracting the collagen concentration per mg of control sample from the concentrations of the various constructs studied (mass in μg of collagen per mg of wet weighed sample at the end of culture cell):

TABLE 4

| | Neosynthesized collagen concentration in |
|---|---|
| Reconstruction conditions | constructs (μg of collagen/mg of sample) |
| Classic bilayer bioprinting | 0.12044817 |
| Hybrid bilayer bioprinting | 0.17182893 |

As the value corresponding to the control sample is non-zero, it can be deduced that the sircol test identifies the gelatin in the FAG hydrogel as collagen. The constructs showed a higher collagen concentration than the control, confirming the presence of neosynthesized collagen.

Histological analyses were performed at the end of culture. The constructs were cut in half. The first half was fixed in 4% formalin for 24 hours, then dehydrated by successive baths of absolute ethanol and methylcyclohexane with an STP 120 dehydrator (Microm) and embedded in kerosene. Sections 5 μm thick were cut using an HM 340e microtome (Microm). Hematoxylin Phloxine Saffron (HPS) staining was then performed on these sections.

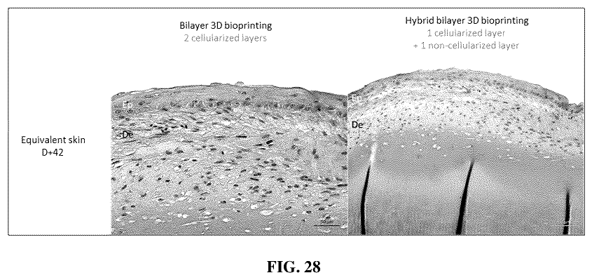

The results are shown in FIG. 28, where the images reveal that both conditions tested supported the establishment of a thick, healthy dermis with extracellular matrix synthesis and a differentiated, pluristratified epidermis.

This study demonstrated the feasibility of creating reconstructed tissue from FAG hydrogel supports consolidated with TAG. Different conditions led to reconstructed skin (thick equivalent dermis and suitably differentiated epidermis). The two methods studied here produced mature dermal-epidermal assemblies demonstrating neosynthesis of collagen.

1-17. (canceled)

18. A method for consolidating a hydrogel comprising alginate and gelatin, said method comprising preparing the hydrogel, and then contacting it with a consolidation solution comprising at least one divalent cation and transglutaminase, and optionally one or more other divalent cations.

19. The method according to claim 18, wherein the contact of the hydrogel with the consolidation solution is carried out by immersion of the hydrogel in the consolidation solution, or by spraying the hydrogel with the consolidation solution.

20. The method according to claim 18, wherein the divalent cation(s) is/are chosen from the group comprising calcium, strontium and barium.

21. The method according to claim 20, wherein the divalent cation is calcium.

22. The method according to claim 18, wherein the hydrogel comprises from 0.5 to 3% of alginate, from 1 to 17.5% of gelatin.

23. The method according to claim 18, wherein the hydrogel further comprises fibrinogen and in that the consolidation solution further comprises thrombin.

24. The method according to claim 23, wherein the hydrogel comprises up to 2% of fibrinogen.

25. The method according to claim 24, wherein the consolidation solution comprises:
from 0.003 to 30% of divalent cation,
from 0.004 to 16% of transglutaminase, and optionally
from 2 to 25 U/ml of thrombin.

26. The method according to claim 18, wherein the hydrogel further comprises living cells integrated during hydrogel preparation prior to its consolidation.

27. The method according to claim 18, wherein the contact with the consolidation solution is carried out:
at a temperature ranging from 15 to 40° C., and/or
for a period ranging from 30 minutes to 6 h.

28. The method according to claim 18, wherein the hydrogel is shaped prior to its consolidation.

29. The method according to claim 28, wherein said shaping is carried out by molding or additive manufacturing technique.

30. A method for consolidating a hydrogel comprising alginate and gelatin, said method comprising contacting a hydrogel with a consolidation solution comprising at least one divalent cation and transglutaminase, and optionally one or more other divalent cations.

31. The method of claim 30, wherein that the at least one divalent cation is calcium.

32. A hydrogel comprising consolidated alginate and gelatin, intended to be implanted in a human subject, the hydrogel being obtainable by a consolidation method according to claim 18, optionally said hydrogel further comprises living cells.

33. A method for consolidating a hydrogel comprising alginate and gelatin, said method comprising, in order, the following steps:
preparation of the hydrogel,
shaping the hydrogel,
contacting the hydrogel with at least one divalent cation and transglutaminase, and optionally one or more other divalent cations.

34. The method according to claim 33, wherein the hydrogel preparation does not require:
the shaping of one or more components prior to the preparation of the hydrogel, and/or
the addition of fibers.

35. The method according to claim 33, wherein the divalent cation(s) is/are chosen from the group comprising calcium, strontium and barium.

36. The method according to claim 35, wherein the divalent cation is calcium.

37. A hydrogel comprising consolidated alginate and gelatin, intended to be implanted in a human subject, the hydrogel being obtainable by a consolidation method according to claim 33, optionally said hydrogel further comprises living cells.
